(19) United States
(12) Patent Application Publication
(10) Pub. No.: US 2015/0275224 A1
(43) Pub. Date: Oct. 1, 2015

(54) PLANTS CONTAINING A HETEROLOGOUS FLAVOHEMOGLOBIN GENE AND METHODS OF USE THEREOF

(71) Applicant: MONSANTO TECHNOLOGY LLC, St. Louis, MO (US)

(72) Inventors: Amarjit Basra, Chesterfield, MO (US); Mike Edgerton, St. Louis, MO (US); Garrett J. Lee, Royersford, PA (US); Maolong Lu, Hillsborough, NJ (US); Linda L. Lutfiyya, St. Louis, MO (US); Wei Wu, Chesterfield, MO (US); Xiaoyun Wu, Chesterfield, MO (US)

(21) Appl. No.: 14/685,442

(22) Filed: Apr. 13, 2015

Related U.S. Application Data
(63) Continuation of application No. 12/897,757, filed on Oct. 4, 2010, now abandoned.
Application No. 12/897,757 is a continuation of application No. 11/429,629, filed on May 5, 2006, now abandoned.
(60) Provisional application No. 60/678,166, filed on May 5, 2005.

(51) Int. Cl. C12N 15/82

(52) U.S. Cl. CPC C12N 15/8251; C12N 15/8261

(57) ABSTRACT

Plant nitrogen use efficiency in corn has been improved by transformation with a flavohemoglobin gene. Plants comprising a flavohemoglobin gene have decreased nitric oxide (NO) levels, increased biomass accumulation under a sufficient nitrogen growth condition, and increased chlorophyll content under a limiting nitrogen growth condition. Additionally, these transformed plants evidence higher levels of yield.

### CROSS REFERENCE TO RELATED APPLICATIONS

This application claims benefit under 35USC §119(e) of U.S. provisional application Ser. No. 60/678,166, filed May 5, 2005, and herein incorporated by reference in its entirety.

### INCORPORATION OF SEQUENCE LISTING

Two copies of the sequence listing (Copy 1 and Copy 2) and a computer readable form (CRF) of the sequence listing, all on CD-R's, each containing the file named 52267B_{—}05052006. ST25.txt, which is 779,000 bytes (measured in MS-WINDOWS) and was created on May 5, 2006, are herein incorporated by reference

### FIELD OF THE INVENTION

Disclosed herein are inventions in the field of plant genetics and developmental biology. More specifically, the present inventions provide transgenic seeds for crops, wherein the genome of said seed comprises recombinant DNA for expression of a heterologous flavohemoglobin protein, which results in the production of transgenic plants with increased growth, yield and/or improved nitrogen use efficiency.

### BACKGROUND OF THE INVENTION

Nitrogen is often the limiting element in plant growth and productivity. Metabolism, growth and development of plants are profoundly affected by their nitrogen supply. Restricted nitrogen supply alters shoot to root ratio, root development, activity of enzymes of primary metabolism and the rate of senescence (death) of older leaves. All field crops have a fundamental dependence on inorganic nitrogenous fertilizer. Since fertilizer is rapidly depleted from most soil types, it must be supplied to growing crops two or three times during the growing season. Nitrogenous fertilizer, which is usually supplied as ammonium nitrate, potassium nitrate, or urea, typically accounts for 40% of the costs associated with crops such as corn and wheat. It has been estimated that approximately 11 million tons of nitrogenous fertilizer are used in both North America and Western Europe annually, costing farmers $2.2 billion each year (Sheldrick, 1987, World Nitrogen Survey, Technical Paper no. 59, Washington, D.C.). Furthermore, World Bank projections suggest that annual nitrogen fertilizer demand worldwide will increase from around 90 million tons to well over 130 million tons over the next ten years. Increased use efficiency of nitrogen by plants should enable crops to be cultivated with lower fertilizer input, or alternatively on soils of poorer quality and would therefore have significant economic impact in both developed and developing agricultural systems.

Using conventional selection techniques, plant breeders have attempted to improve nitrogen use efficiency by exploiting the variation available in natural populations of corn, wheat, rice and other crop species. There are, however, considerable difficulties associated with the screening of extensive populations in conventional breeding programs for traits which are difficult to assess under field conditions, and such selection strategies have been largely unsuccessful. Recent advances in genetic engineering have provided the prerequisite tools to transform plants to contain foreign (often referred to as “heterogenous or heterologous”) or improved endogenous genes. The ability to introduce specific DNA into plant genomes provides further opportunities for generation of plants with improved and/or unique phenotypes.

Flavohemoglobins, composed of a heme-binding domain and a ferredoxin reductase-like domain, detoxify high levels of nitric oxide (NO) through oxygenation of NO to NO₃⁻, functioning as an NO dioxygenase (NOD) in Escherichia coli (Vasudevan et al., 1991, Mol. Gen. Genet. 226: 49-58, and Gardener et al., 2002, J. of Biological Chemistry 270: 8166-8171).

It has been reported that NO can participate in many physiological responses in plants, including pathogen response, programmed cell death, germination (Beligni and Lamattina, 2000, Planta. 210: 215-221), phytoalexin production (Noritake, et al., 1996), and ethylene emission (Leshem, 2000, J. Exp. Bot. 51: 1471-1473). In addition, NO was found to have a critical role in salicylic acid signaling (Klessig, et al., 2000, Proc. Natl. Acad. Sci. USA. 97: 8849-8855), and cytokinin signaling. It was found that NO gives rise to parallel signaling pathways through increased nitric oxide synthase (NOS, EC1.14.13.39) activity, which mediate responses of specific genes to UV-B tolerance. Furthermore, nitric oxide has been reported to mediate photomorphogenic responses in wheat, lettuce, potato and A. thaliana, promote root elongation in corn (Gouvea, 1997, Plant Growth Regulation 21: 183-187), and promote ripening in strawberry and avocado (Leshem and Pinchasov, 2000, J. Exp. Bot. 51:1471-1473). Involvement of NO in the tobacco defense response is perhaps the best documented role played by nitric oxide in plant signaling (Klessig, et al., 2000, Proc. Natl, Acad. Sci USA 97:8849-8855; Foissner, et al., 2000, Plant J. 23: 817-824).

We thus contemplated that the removal of endogenous NO by overexpression of NO detoxifying enzymes can uncover what role(s) NO plays in the expression of agronomic traits in corn, such as kernel maturation, leaf senescence, disease resistance, root growth and/or photomorphogensis. Overexpression of enzymes activated by NO may also affect similar processes. In both cases agronomic traits may also be improved by either a reduction in nitrosative stress or an amplification of NO signaling. The present invention is based, in part, on our surprising finding that expression of an E. coli flavohemoglobin in corn plants resulted in more robust growth characteristics under either sufficient or limiting nitrogen growth conditions, and increased seed yield.

### SUMMARY OF THE INVENTION

The present invention is directed to seed from a transgenic plant line, wherein said seed comprises in its genome a recombinant polynucleotide providing for expression of a flavohemoglobin protein. Of particular interest, the present invention provides transgenic seed containing a flavohemoglobin protein to produce transgenic plants having improved agronomic traits. The improved agronomic traits are characterized as a faster growth rate, increased fresh or dry biomass, increased seed or fruit yield, increased seed or fruit nitrogen content, increased free amino acid content in seed or fruit, increased protein content in seed or fruit, and/or increased protein content in vegetative tissue under a sufficient nitrogen growth condition or a limiting nitrogen growth condition. Also of particular interest in the present invention is seed from transgenic crop plants, preferably maize (corn—Zea mays) or soybean (soy—Glycine max) plants. Other plants of interest in the present invention for production of transgenic seed comprising a heterologous flavohemoglobin gene include, without limitation, cotton, canola, wheat, sunflower, sorghum, alfalfa, barley, millet, rice, tobacco, fruit and vegetable crops, and turfgrass.

Therefore, in accomplishing the above, the present invention, in one aspect, provides three non-naturally occurring polynucleotides, as set forth in SEQ ID NO 1, 2, and 260 with optimized plant expression codons for expressing E. coli HMP protein, Yeast YHB1 protein and Erwinia flavohemoglobin protein in plants respectively. The present invention further provides recombinant DNA constructs for plant transformation containing a flavohemoglobin gene under the control of a promoter for plant expression.

The present invention, in another aspect, provides the methods of generating a transgenic plant having improved agronomic traits including a faster growth rate, increased fresh or dry biomass, increased seed or fruit yield, increased seed or fruit nitrogen content, increased free amino acid content in seed or fruit, increased protein content in seed or fruit, and/or increased protein content in vegetative tissue. The method comprises the steps of transforming a plant cell with a recombinant DNA construct for expression of a flavohemoglobin protein, regenerating the transformed plant cell into a transgenic plant expressing the flavohemoglobin protein, and screening to identify a plant having improved agronomic traits. The improved agronomic traits are characterized as a faster growth rate, increased growth rate, increased seed or fruit nitrogen content, increased free amino acid content in seed or fruit, and/or increased protein content in vegetative tissue either under a sufficient nitrogen growth condition or a limiting nitrogen condition.

The present invention, in yet another aspect, provides exemplary flavohemoglobin proteins identified as homologs of E. Coli HMP as set forth in SEQ ID NO: 130 through SEQ ID NO: 256, which can be used to practice the present invention.

### BRIEF DESCRIPTION OF THE DRAWINGS AND SEQUENCE LISTING

FIG. 1. Molecular function of a flavohemoglobin protein in a plant cell

FIG. 2. Recombinant DNA construct pMON69471 comprising SEQ ID NO:3 for plant transformation

FIG. 3. Recombinant DNA construct pMON67827 comprising SEQ ID NO:4 for plant transformation

FIG. 4. Recombinant DNA construct pMON95605 comprising SEQ ID NO:105 for plant transformation

FIG. 5. Corn transformation construct pMON99286 for expression of codon optimized E. coli HMP gene

FIG. 6. Corn transformation construct pMON99261 for expression of codon optimized E. coli HMP gene

FIG. 7. Corn transformation construct pMON99276 for expression of codon optimized E. coli HMP gene

FIG. 8. Corn transformation construct pMON94446 for expression of E. coli HMP gene

FIG. 9. Corn transformation construct pMON102760 for expression of Yeast YHB gene

FIG. 10. Soybean transformation construct pMON95622 for expression of E. coli HMP gene

SEQ ID NO:1, the codon optimized E. coli HMP gene
SEQ ID NO:2, the codon optimized Yeast YHB gene
SEQ ID NO:3, E. coli HMP gene
SEQ ID NO:4: Yeast YHB gene
SEQ ID NO:5, E. coli HMP protein
SEQ ID NO:6, Yeast YHB protein
SEQ ID NO:7 through SEQ ID NO: 129, DNA sequences of E. coli HMP homologs
SEQ ID NO:130 though SEQ ID NO:256, protein sequences of E. coli HMP homologs

TABLE 1
The following table lists a DNA sequence identified as NUC
SEQ ID NO and the flavohemoglobin protein sequence, encoded
by the corresponding DNA, identified by PEP SEQ ID NO.

| NUC SEQ ID | | NUC SEQ ID | | NUC SEQ ID | | NUC SEQ ID | |
|---|---|---|---|---|---|---|---|
| encodes PEP | | encodes PEP | | encodes PEP | | encodes PEP | |
| SEQ ID | | SEQ ID | | SEQ ID | | SEQ ID | |
| NUC | PEP | NUC | PEP | NUC | PEP | NUC | PEP |
| SEQ | SEQ | SEQ | SEQ | SEQ | SEQ | SEQ | SEQ |
| ID | ID | ID | ID | ID | ID | ID | ID |
| 1 | 5 | 2 | 6 | 3 | 5 | 4 | 6 |
| 7 | 130 | 37 | 161 | 68 | 192 | 99 | 223 |
| 8 | 131 | 38 | 162 | 69 | 193 | 100 | 226 |
| 9 | 132 | 39 | 163 | 70 | 194 | 101 | 227 |
| 10 | 133 | 40 | 164 | 71 | 195 | 102 | 228 |
| 11 | 134 | 41 | 165 | 72 | 196 | 103 | 230 |
| 12 | 135 | 42 | 166 | 73 | 197 | 104 | 231 |
| 13 | 136 | 43 | 167 | 74 | 198 | 105 | 232 |
| 14 | 137 | 44 | 168 | 75 | 199 | 106 | 233 |
| 15 | 138 | 45 | 169 | 76 | 200 | 107 | 234 |
| 16 | 140 | 46 | 170 | 77 | 201 | 108 | 235 |
| 17 | 141 | 47 | 171 | 78 | 202 | 109 | 236 |
| 18 | 142 | 48 | 172 | 79 | 203 | 110 | 237 |
| 19 | 143 | 49 | 173 | 80 | 204 | 111 | 238 |
| 20 | 144 | 50 | 174 | 81 | 205 | 112 | 239 |
| 21 | 145 | 51 | 175 | 82 | 206 | 113 | 240 |
| 22 | 146 | 52 | 176 | 83 | 207 | 114 | 241 |
| 23 | 147 | 53 | 177 | 84 | 208 | 115 | 242 |
| 24 | 148 | 54 | 178 | 85 | 209 | 116 | 243 |
| 25 | 149 | 55 | 179 | 86 | 210 | 117 | 244 |
| 26 | 150 | 56 | 180 | 87 | 211 | 118 | 245 |
| 27 | 151 | 57 | 181 | 88 | 212 | 119 | 246 |
| 28 | 152 | 58 | 182 | 89 | 213 | 120 | 247 |
| 29 | 153 | 59 | 183 | 90 | 214 | 121 | 248 |
| 30 | 154 | 60 | 184 | 91 | 215 | 122 | 249 |
| 31 | 155 | 61 | 185 | 92 | 216 | 123 | 250 |
| 32 | 156 | 62 | 186 | 93 | 217 | 124 | 251 |
| 33 | 157 | 63 | 187 | 94 | 218 | 125 | 252 |
| 34 | 158 | 64 | 188 | 95 | 219 | 126 | 253 |
| 35 | 159 | 65 | 189 | 96 | 220 | 127 | 254 |
| 36 | 160 | 66 | 190 | 97 | 221 | 128 | 255 |
| 37 | 161 | 67 | 191 | 98 | 222 | 129 | 256 |

SEQ ID NO: 257, the full length sequence of recombinant DNA construct pMON69471
SEQ ID NO: 258, the full length sequence of recombinant DNA construct pMON67827
SEQ ID NO: 259, the full length sequence of recombinant DNA construct pMON95605
SEQ ID NO: 260, the codon optimized HMP gene from Erwinia carotovora
SEQ ID NO: 261, the full length sequence of recombinant DNA construct pMON99286
SEQ ID NO: 262, the full length sequence of recombinant DNA construct pMON99261
SEQ ID NO: 263, the full length sequence of recombinant DNA construct pMON99276
SEQ ID NO: 264, the full length sequence of recombinant DNA construct pMON94446
SEQ ID NO: 265, the full length sequence of recombinant DNA construct pMON102760
SEQ ID NO: 266, the full length sequence of recombinant DNA construct pMON95622
SEQ ID NO: 267 through SEQ ID NO: 272: PCR primers.

### DETAILED DESCRIPTION OF THE INVENTION

The present invention is directed to transgenic plant seed, wherein the genome of said transgenic plant seed comprises a recombinant DNA encoding a flavohemoglobin, as provided herein, and transgenic plant grown from such seed. Transgenic plant provided by the present invention possesses an improved trait as compared to the trait of a control plant under either limited nitrogen growth condition or sufficient nitrogen growth condition. Of particular interest are the transgenic plants grown from transgenic seeds provided herein wherein the improved trait is increased seed yield. Recombinant DNA constructs disclosed by the present invention comprise recombinant DNA providing for the production of mRNA to modulate gene expression, imparting improved traits to plants.

As used herein, “flavohemoglobin” refers to a protein that is composed of a heme binding domain and a ferredoxin reductase-like FAD- and NAD-binding domain. It is also known as flavohemoprotein, nitric oxide dioxygenase, nitric oxide oxygenase and flavodoxin reductase. Flavohemoglobin genes from E. coli, A. eutrophus, Saccharomyces cerevisiae and Vitreoscilla sp are abbreviated as HMP, FHP, YHB1 (or YHG), and VI-IP respectively.

As used herein, “gene” refers to chromosomal DNA, plasmid DNA, cDNA, synthetic DNA, or other DNA that encodes a peptide, polypeptide, protein, or RNA molecule, and regions flanking the coding sequences involved in the regulation of expression.

As used herein, “transgenic seed” refers to a plant seed whose genome has been altered by the incorporation of recombinant DNA, e.g., by transformation as described herein. The term “transgenic plant” is used to refer to the plant produced from an original transformation event, or progeny from later generations or crosses of a plant to a transformed plant, so long as the progeny contains the recombinant DNA in its genome.

As used herein, “recombinant DNA” refers to a polynucleotide having a genetically engineered modification introduced through combination of endogenous and/or exogenous elements in a transcription unit, manipulation via mutagenesis, restriction enzymes, and the like or simply by inserting multiple copies of a native transcription unit. Recombinant DNA may comprise DNA segments obtained from different sources, or DNA segments obtained from the same source, but which have been manipulated to join DNA segments which do not naturally exist in the joined form. A recombinant polynucleotide may exist outside of the cell, for example as a PCR fragment, or integrated into a genome, such as a plant genome.

As used herein, “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g., by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance.

As used herein, “control plant” is a plant without recombinant DNA disclosed herein. A control plant is used to measure and compare trait improvement in a transgenic plant with such recombinant DNA. A suitable control plant may be a non-transgenic plant of the parental line used to generate a transgenic plant herein. Alternatively, a control plant may be a transgenic plant that comprises an empty vector or marker gene, but does not contain the recombinant DNA that produces the trait improvement. A control plant may also be a negative segregant progeny of hemizygous transgenic plant.

As used herein, “improved trait” refers to a trait with a detectable improvement in a transgenic plant relative to a control plant or a reference. In some cases, the trait improvement can be measured quantitatively. For example, the trait improvement can entail at least a 2% desirable difference in an observed trait, at least a 5% desirable difference, at least about a 10% desirable difference, at least about a 20% desirable difference, at least about a 30% desirable difference, at least about a 50% desirable difference, at least about a 70% desirable difference, or at least about a 100% difference, or an even greater desirable difference. In other cases, the trait improvement is only measured qualitatively. It is known that there can be a natural variation in a trait. Therefore, the trait improvement observed entails a change of the normal distribution of the trait in the transgenic plant compared with the trait distribution observed in a control plant or a reference, which is evaluated by statistical methods provided herein. Trait improvement includes, but not limited to, yield increase, including increased yield under non-stress conditions and increased yield under environmental stress conditions. Stress conditions may include, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density.

Many agronomic traits can affect “yield”, including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Other traits that can affect yield include, efficiency of germination (including germination in stressed conditions), growth rate (including growth rate in stressed conditions), ear number, seed number per ear, seed size, composition of seed (starch, oil, protein) and characteristics of seed fill. Also of interest is the generation of transgenic plants that demonstrate desirable phenotypic properties that may or may not confer an increase in overall plant yield. Such properties include enhanced plant morphology, plant physiology or improved components of the mature seed harvested from the transgenic plant.

As used herein, “sufficient nitrogen growth condition” refers to the growth condition where the soil or growth medium contains or receives enough amounts of nitrogen nutrient to sustain a healthy plant growth and/or for a plant to reach its typical yield for a particular plant species or a particular strain. Sufficient nitrogen growth conditions vary between species and for varieties within a species, and also vary between different geographic locations. However, one skilled in the art knows what constitute nitrogen non-limiting growth conditions for the cultivation of most, if not all, important crops, in a specific geographic location. For example, for the cultivation of wheat see Alcoz, et al., Agronomy Journal 85:1198-1203 (1993), Rao and Dao, J. Am. Soc. Agronomy 84:1028-1032 (1992), Howard and Lessman, Agronomy Journal 83:208-211 (1991); for the cultivation of corn see Wood, et al., J. of Plant Nutrition 15: 487-500 (1992), Tollenear, et al., Agronomy Journal 85:251-255 (1993), Straw, et al., Tennessee Farm and Home Science: Progress Report, 166:20-24 (Spring 1993), Dara, et al., J. Am. Soc. Agronomy 84:1006-1010 (1992), Binford, et al., Agronomy Journal 84:53-59 (1992); for the cultivation of soybean see Chen, et al., Canadian Journal of Plant Science 72:1049-1056 (1992), Wallace, et al. Journal of Plant Nutrition 13:1523-1537 (1990); for the cultivation of rice see Oritani and Yoshida, Japanese Journal of Crop Science 53:204-212 (1984); for the cultivation of tomato see Grubinger, et al., Journal of the American Society for Horticultural Science 118:212-216 (1993), Cerne, M., Acta Horticulture 277:179-182, (1990); for the cultivation of pineapple see Asoegwu, S. N., Fertilizer Research 15:203-210 (1988), Asoegwu, S. N., Fruits 42:505-509 (1987), for the cultivation of lettuce see Richardson and Hardgrave, Journal of the Science of Food and Agriculture 59:345-349 (1992); for the cultivation of potato see Porter and Sisson, American Potato Journal, 68:493-505 (1991); for the cultivation of brassica crops see Rahn, et al., Conference “Proceedings, second congress of the European Society for Agronomy” Warwick Univ., p. 424-425 (Aug. 23-28, 1992); for the cultivation of banana see Hegde and Srinivas, Tropical Agriculture 68:331-334 (1991), Langenegger and Smith, Fruits 43:639-643 (1988); for the cultivation of strawberries see Human and Kotze, Communications in Soil Science and Plant Analysis 21:771-782 (1990); for the cultivation of sorghum see Mahalle and Seth, Indian Journal of Agricultural Sciences 59:395-397 (1989); for the cultivation of sugar can e see Yadav, R. L., Fertiliser News 31:17-22 (1986), Yadav and Sharma, Indian Journal of Agricultural Sciences 53:38-43 (1983); for the cultivation of sugar beet see Draycott, et al., Conference “Symposium Nitrogen and Sugar Beet” International Institute for Sugar Beet Research—Brussels Belgium, p. 293-303 (1983). See also Goh and Haynes, “Nitrogen and Agronomic Practice” in Mineral Nitrogen in the Plant-Soil System, Academic Press, Inc., Orlando, Fla., p. 379-468 (1986), Engelstad, 0. P., Fertilizer Technology and Use, Third Edition, Soil Science Society of America, p. 633 (1985), Yadav and Sharmna, Indian Journal of Agricultural Sciences, 53:3-43 (1983).

As used herein, “nitrogen nutrient” means any one or any mix of the nitrate salts commonly used as plant nitrogen fertilizer, including, but not limited to, potassium nitrate, calcium nitrate, sodium nitrate, ammonium nitrate. The term ammonium as used herein means any one or any mix of the ammonium salts commonly used as plant nitrogen fertilizer, e.g., ammonium nitrate, ammonium chloride, ammonium sulfate, etc. One skilled in the art would recognize what constitute such soil, media and fertilizer inputs for most plant species.

“Limiting nitrogen growth condition” used herein refers to a plant growth condition that does not contain sufficient nitrogen nutrient to maintain a healthy plant growth and/or for a plant to reach its typical yield under a sufficient nitrogen growth condition. For example, a limiting nitrogen condition can refers to a growth condition with 50% or less of the conventional nitrogen inputs.

As used herein, “increased yield” of a transgenic plant of the present invention may be evidenced and measured in a number of ways, including test weight, seed number per plant, seed weight, seed number per unit area (i.e., seeds, or weight of seeds, per acre), bushels per acre, tons per acre, kilo per hectare. For example, maize yield may be measured as production of shelled corn kernels per unit of production area, e.g., in bushels per acre or metric tons per hectare, often reported on a moisture adjusted basis, e.g., at 15.5% moisture. Increased yield may result from improved utilization of key biochemical compounds, such as nitrogen, phosphorous and carbohydrate, or from improved tolerance to environmental stresses, such as cold, heat, drought, salt, and attack by pests or pathogens. Trait-improving recombinant DNA may also be used to provide transgenic plants having improved growth and development, and ultimately increased yield, as the result of modified expression of plant growth regulators or modification of cell cycle or photosynthesis pathways.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissues are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions, or certain chemicals, or the presence of light. Tissue specific, tissue preferred, cell type specific, and inducible promoters constitute the class of “non-constitutive” promoters. A “constitutive” promoter is a promoter which is active under most conditions. As used herein, “antisense orientation” includes reference to a polynucleotide sequence that is operably linked to a promoter in an orientation where the antisense strand is transcribed. The antisense strand is sufficiently complementary to an endogenous transcription product such that translation of the endogenous transcription product is often inhibited. As used herein, “operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

As used herein, “consensus sequence” refers to an artificial, amino acid sequence of conserved parts of the proteins encoded by homologous genes, e.g., as determined by a CLUSTALW alignment of amino acid sequence of homolog proteins.

Homologous genes are genes related to a second gene, which encode proteins with the same or similar biological function to the protein encoded by the second gene. Homologous genes may be generated by the event of speciation (see ortholog) or by the event of genetic duplication (see paralog). “Orthologs” refer to a set of homologous genes in different species that evolved from a common ancestral gene by specification. Normally, orthologs retain the same function in the course of evolution; and “paralogs” refer to a set of homologous genes in the same species that have diverged from each other as a consequence of genetic duplication. Thus, homologous genes can be from the same or a different organism. As used herein, “homolog” means a protein that performs the same biological function as a second protein including those identified by sequence identity search.

Percent identity refers to the extent to which two optimally aligned DNA or protein segments are invariant throughout a window of alignment of components, e.g., nucleotide sequence or amino acid sequence. An “identity fraction” for aligned segments of a test sequence and a reference sequence is the number of identical components which are shared by sequences of the two aligned segments divided by the total number of sequence components in the reference segment over a window of alignment which is the smaller of the full test sequence or the full reference sequence. “Percent identity” (“% identity”) is the identity fraction times 100. “% identity to a consensus amino acid sequence” is 100 times the identity fraction in a window of alignment of an amino acid sequence of a test protein optimally aligned to consensus amino acid sequence of this invention.

### Recombinant DNA Constructs

As used herein, “expression” refers to transcription of DNA to produce RNA. The resulting RNA may be without limitation mRNA encoding a protein, antisense RNA that is complementary to an mRNA encoding a protein, or an RNA transcript comprising a combination of sense and antisense gene regions, such as for use in RNAi technology. Expression as used herein may also refer to production of encoded protein from mRNA. “Ectopic expression” refers to the expression of an RNA molecule or a protein in a cell type other than a cell type in which the RNA or the protein is normally expressed, or at a time other than a time at which the RNA or the protein is normally expressed, or at a expression level other than the level at which the RNA normally is expressed. “Overexpression” used herein indicates that the expression level of a target protein, in a transgenic plant or in a host cell of the transgenic plant, exceeds levels of expression in a non-transgenic plant. In a preferred embodiment of the present invention, a recombinant DNA construct comprises the polynucleotide of interest in the sense orientation relative to the promoter to achieve gene overexpression.

The present invention provides recombinant DNA constructs comprising a polynucleotide disclosed herein, which encodes for a flavohemoglobin protein. Such constructs also typically comprise a promoter operatively linked to said polynucleotide to provide for expression in a target plant. Other construct components may include additional regulatory elements, such as 5′ or 3′ untranslated regions (such as polyadenylation sites), intron regions, and transit or signal peptides.

In a preferred embodiment, a polynucleotide of the present invention is operatively linked in a recombinant DNA construct to a promoter functional in a plant to provide for expression of the polynucleotide in the sense orientation such that a desired polypeptide is produced to achieve overexpression or ectopic expression.

Recombinant constructs prepared in accordance with the present invention may also generally include a 3′ untranslated DNA region (UTR) that typically contains a polyadenylation sequence following the polynucleotide coding region. Examples of useful 3′ UTRs include those from the nopaline synthase gene of Agrobacterium tumefaciens (nos), a gene encoding the small subunit of a ribulose-1,5-bisphosphate carboxylase-oxygenase (rbcS), and the T7 transcript of Agrobacterium tumefaciens. Constructs and vectors may also include a transit peptide for targeting of a gene target to a plant organelle, particularly to a chloroplast, leucoplast or other plastid organelle. For descriptions of the use of chloroplast transit peptides, see U.S. Pat. No. 5,188,642 and U.S. Pat. No. 5,728,925, incorporated herein by reference.

The recombinant DNA construct may include other elements. For example, the construct may contain DNA segments that provides replication function and antibiotic selection in bacterial cells. For example, the construct may contain an E. coli origin of replication such as ori322 or a broad host range origin of replication such as oriV, oriRi or oriColE.

The construct may also comprise a selectable marker such as an Ec-ntpll-Tn5 that encodes a neomycin phosphotransferase II gene obtained from Tn5 conferring resistance to a neomycin and kanamysin, Spc/Str that encodes for Tn7 aminoglycoside adenyltransferase (aadA) conferring resistance to spectinomycin or streptomycin, or a gentamicin (Gm, Gent) or one of many known selectable marker gene.

The vector or construct may also include a screenable marker and other elements as appropriate for selection of plant or bacterial cells having DNA constructs of the invention. DNA constructs are designed with suitable selectable markers that can confer antibiotic or herbicide tolerance to the cell. The antibiotic tolerance polynucleotide sequences include, but are not limited to, polynucleotide sequences encoding for proteins involved in tolerance to kanamycin, neomycin, hygromycin, and other antibiotics known in the art. An antibiotic tolerance gene in such a vector may be replaced by herbicide tolerance gene encoding for 5-enolpyruvylshikimate-3-phosphate synthase (EPSPS, described in U.S. Pat. Nos. 5,627,061, and 5,633,435; Padgette, et al. Herbicide Resistant Crops, Lewis Publishers, 53-85, 1996; and in Penaloza-Vazquez, et al., Plant Cell Reports 14:482-487, 1995) and aroA (U.S. Pat. No. 5,094,945) for glyphosate tolerance, bromoxynil nitrilase (Bxn) for Bromoxynil tolerance (U.S. Pat. No. 4,810,648), phytoene desaturase (crtI (Misawa, et al., Plant J. 4:833-840, 1993; and Misawa, et al., Plant J. 6:481-489, 1994) for tolerance to norflurazon, acetohydroxyacid synthase (AHAS, Sathasiivan, et al., Nucl. Acids Res. 18:2188-2193, 1990). Herbicides for which transgenic plant tolerance has been demonstrated and for which the method of the present invention can be applied include, but are not limited to: glyphosate, sulfonylureas, imidazolinones, bromoxynil, delapon, cyclohezanedione, protoporphyrionogen oxidase inhibitors, and isoxaslutole herbicides.

Other examples of selectable markers, screenable markers and other elements are well known in the art and may be readily used in the present invention. Those skilled in the art should refer to the following for details (for selectable markers, see Potrykus, et al., Mol. Gen. Genet. 199:183-188, 1985; Hinchee, et al., Bio. Techno. 6:915-922, 1988; Stalker, et al., J. Biol. Chem. 263:6310-6314, 1988; European Patent Application 154,204; Thillet, et al., J. Biol. Chem. 263:12500-12508, 1988; for screenable markers see, Jefferson, Plant Mol. Biol, Rep. 5: 387-405, 1987; Jefferson, et al., EMBO J. 6: 3901-3907, 1987; Sutcliffe, et al., Proc. Natl. Acad. Sci. U.S.A. 75: 3737-3741, 1978; Ow, et al., Science 234: 856-859, 1986; Ikatu, et al., Bio. Technol. 8: 241-242, 1990; and for other elements see, European Patent Application Publication Number 0218571; Koziel et al., Plant Mol. Biol. 32: 393-405; 1996).

In one embodiment of the present invention, recombinant DNA constructs also include a transit peptide for targeting of a gene target to a plant organelle, particularly to a chloroplast, leucoplast or other plastid organelle. For descriptions of the use of chloroplast transit peptides see U.S. Pat. No. 5,188,642 and U.S. Pat. No. 5,728,925, incorporated herein by reference. For description of the transit peptide region of an Arabidopsis EPSPS gene useful in the present invention, see Klee, H. J. et al., (MGG (1987) 210:437-442).

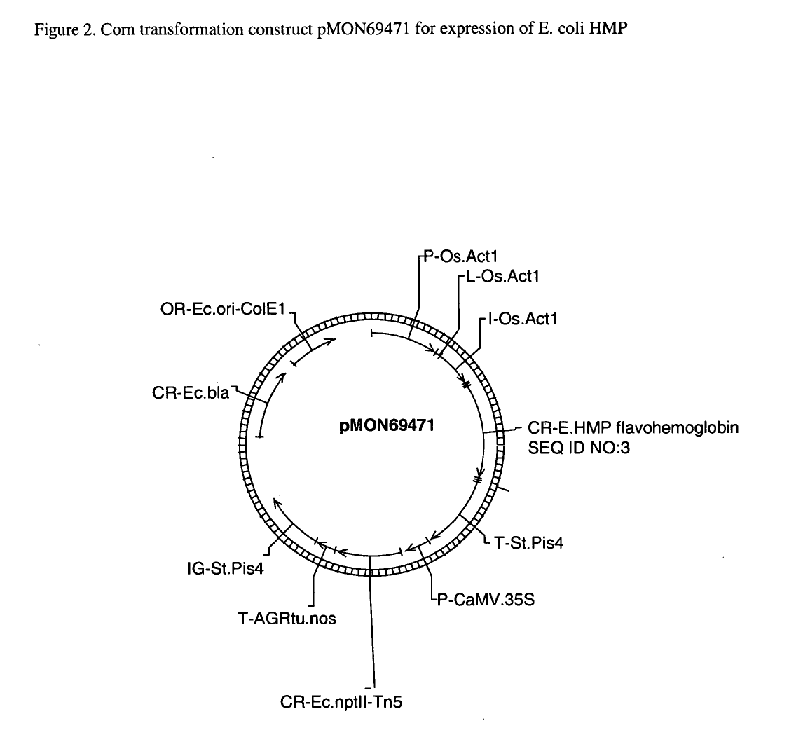

The essential components of the expression cassette in the recombinant DNA construct of the present invention are operably linked with each other in a specific order to cause the expression of the desired gene product, i.e., flavohemoglobin protein, in a plant. Specific orders of operably linked essential components of the expression vectors are illustrated in FIG. 2-4.

### Recombinant DNA and Polynucleotides

As used herein, both terms “a coding sequence” and “a coding polynucleotide molecule” mean a polynucleotide molecule that can be translated into a polypeptide, usually via mRNA, when placed under the control of appropriate regulatory molecules. The boundaries of the coding sequence are determined by a translation start codon at the 5′-terminus and a translation stop codon at the 3′-terminus. A coding sequence can include, but is not limited to, genomic DNA, cDNA, and chimeric polynucleotide molecules. A coding sequence can be an artificial DNA. An artificial DNA, as used herein means a DNA polynucleotide molecule that is non-naturally occurring.

Exemplary polynucleotides comprising a coding sequence for a flavohemoglobin for use in the present invention to improve traits in plants are provided herein as SEQ ID NO: 3 and SEQ ID NO: 4, as well as the homologs of such DNA molecules. A subset of the exemplary DNA includes fragments of the disclosed full polynucleotides consisting of oligonucleotides of at least 15, preferably at least 16 or 17, more preferably at least 18 or 19, and even more preferably at least 20 or more, consecutive nucleotides. Such oligonucleotides are fragments of the larger molecules having a sequence selected from the group consisting of SEQ ID NO: 1 through SEQ ID NO: 4, and SEQ ID NO: 7 through SEQ ID NO: 129, and find use, for example, as probes and primers for detection of the polynucleotides of the present invention.

Also of interest in the present invention are variants of the DNA provided herein. Such variants may be naturally occurring, including DNA from homologous genes from the same or a different species, or may be non-natural variants, i.e. an artificial DNA, for example DNA synthesized using chemical synthesis methods, or generated using recombinant DNA techniques. Degeneracy of the genetic code provides the possibility to substitute at least one base of the protein encoding sequence of a gene with a different base without causing the amino acid sequence of the polypeptide produced from the gene to be changed. Hence, a DNA useful in the present invention may have any base sequence that has been changed from the sequences provided herein by substitution in accordance with degeneracy of the genetic code. Artificial DNA molecules can be designed by a variety of methods, such as, methods known in the art that are based upon substituting the codon(s) of a first polynucleotide to create an equivalent, or even an improved, second-generation artificial polynucleotide, where this new artificial polynucleotide is useful for enhanced expression in transgenic plants. The design aspect often employs a codon usage table. The table is produced by compiling the frequency of occurrence of codons in a collection of coding sequences isolated from a plant, plant type, family or genus. Other design aspects include reducing the occurrence of polyadenylation signals, intron splice sites, or long AT or GC stretches of sequence (U.S. Pat. No. 5,500,365, specifically incorporated herein by reference in its entirety). Full length coding sequences or fragments thereof can be made of artificial DNA using methods known to those skilled in the art. Such exemplary artificial DNA molecules provided by the present invention are set forth as SEQ ID NO: 1, 2 and 260.

Homologs of the genes providing DNA demonstrated as useful in improving traits in model plants disclosed herein will generally demonstrate significant identity with the DNA provided herein. DNA is substantially identical to a reference DNA if, when the sequences of the polynucleotides are optimally aligned there is about 60% nucleotide equivalence; more preferably 70%; more preferably 80% equivalence; more preferably 85% equivalence; more preferably 90%; more preferably 95%; and/or more preferably 98% or 99% equivalence over a comparison window. A comparison window is preferably at least 50-100 nucleotides, and more preferably is the entire length of the polynucleotide provided herein. Optimal alignment of sequences for aligning a comparison window may be conducted by algorithms; preferably by computerized implementations of these algorithms (for example, the Wisconsin Genetics Software Package Release 7.0-10.0, Genetics Computer Group, 575 Science Dr., Madison, Wis.). The reference polynucleotide may be a full-length molecule or a portion of a longer molecule. Preferentially, the window of comparison for determining polynucleotide identity of protein encoding sequences is the entire coding region.

### Polypeptides and Proteins

Polypeptides provided by the present invention are entire proteins or at least a sufficient portion of the entire protein to impart the relevant biological activity of the protein. The term “protein” also includes molecules consisting of one or more polypeptide chains. Thus, a protein useful in the present invention may constitute an entire protein having the desired biological activity, or may constitute a portion of an oligomeric protein having multiple polypeptide chains. Proteins useful for generation of transgenic plants having improved traits include the proteins with an amino acid sequence provided herein as SEQ ID NO: 5 and 6, as well as homologs of such proteins.

Homologs of the proteins useful in the present invention may be identified by comparison of the amino acid sequence of the protein to amino acid sequences of proteins from the same or different plant sources, e.g. manually or by using known homology-based search algorithms such as those commonly known and referred to as BLAST, FASTA, and Smith-Waterman. As used herein, a homolog is a protein from the same or a different organism that performs the same biological function as the polypeptide to which it is compared. An orthologous relation between two organisms is not necessarily manifest as a one-to-one correspondence between two genes, because a gene can be duplicated or deleted after organism phylogenetic separation, such as speciation. For a given protein, there may be no ortholog or more than one ortholog. Other complicating factors include alternatively spliced transcripts from the same gene, limited gene identification, redundant copies of the same gene with different sequence lengths or corrected sequence. A local sequence alignment program, e.g. BLAST, can be used to search a database of sequences to find similar sequences, and the summary Expectation value (E-value) used to measure the sequence base similarity. As a protein hit with the best E-value for a particular organism may not necessarily be an ortholog or the only ortholog, a reciprocal BLAST search is used in the present invention to filter hit sequences with significant E-values for ortholog identification. The reciprocal BLAST entails search of the significant hits against a database of amino acid sequences from the base organism that are similar to the sequence of the query protein. A hit is a likely ortholog, when the reciprocal BLAST's best hit is the query protein itself or a protein encoded by a duplicated gene after speciation. Thus, homolog is used herein to described proteins that are assumed to have functional similarity by inference from sequence base similarity.

A further aspect of the invention comprises functional homolog proteins which differ in one or more amino acids from those of a trait-improving protein disclosed herein as the result of one or more of the well-known conservative amino acid substitutions, e.g. valine is a conservative substitute for alanine and threonine is a conservative substitute for serine. Conservative substitutions for an amino acid within the native sequence can be selected from other members of a class to which the naturally occurring amino acid belongs. Representative amino acids within these various classes include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Naturally conservative amino acids substitution groups are: valine-leucine, valine-isoleucine, phenylalanine-tyrosine, lysine-arginine, alanine-valine, aspartic acid-glutamic acid, and asparagine-glutamine. A further aspect of the invention comprises proteins that differ in one or more amino acids from those of a described protein sequence as the result of deletion or insertion of one or more amino acids in a native sequence.

Homologs disclosed provided herein will generally demonstrate significant sequence identity. Of particular interest are proteins having at least 50% sequence identity, more preferably at least about 70% sequence identity or higher, e.g. at least about 80% sequence identity with an amino acid sequence of SEQ ID NO: 5 or 6. Of course useful proteins also include those with higher identity, e.g. 90% to 99% identity. Identity of protein homologs is determined by optimally aligning the amino acid sequence of a putative protein homolog with a defined amino acid sequence and by calculating the percentage of identical and conservatively substituted amino acids over the window of comparison. The window of comparison for determining identity can be the entire amino acid sequence disclosed herein, e.g. the full sequence of any of SEQ ID NO: 5 and 6.

Genes that are homologous to each other can be grouped into families and included in multiple sequence alignments. Then a consensus sequence for each group can be derived. This analysis enables the derivation of conserved and class-(family) specific residues or motifs that are functionally important. These conserved residues and motifs can be further validated with 3D protein structure if available. The consensus sequence can be used to define the full scope of the invention, e.g. to identify proteins with a homolog relationship.

### Promoters

The promoter that causes expression of an RNA that is operably linked to the polynucleotide molecule in a construct usually controls expression pattern of translated polypeptide in a plant. Promoters for practicing the invention may be obtained from various sources including, but not limited to, plants and plant viruses. Several promoters, including constitutive promoters, inducible promoters and tissue-specific promoters, tissue enhanced promoters that are active in plant cells have been described in the literature. It is preferred that the particular promoter selected should be capable of causing sufficient expression to result in the production of an effective amount of a polypeptide to cause the desired phenotype. “Gene overexpression” used herein in reference to a polynucleotide or polypeptide indicates that the expression level of a target protein, in a transgenic plant or in a host cell of the transgenic plant, exceeds levels of expression in a non-transgenic plant. In a preferred embodiment of the present invention, a recombinant DNA construct comprises the polynucleotide of interest in the sense orientation relative to the promoter to achieve gene overexpression.

In accordance with the current invention, constitutive promoters are active under most environmental conditions and states of development or cell differentiation. These promoters are likely to provide expression of the polynucleotide sequence at many stages of plant development and in a majority of tissues. A variety of constitutive promoters are known in the art. Examples of constitutive promoters that are active in plant cells include but are not limited to the nopaline synthase (NOS) promoters; the cauliflower mosaic virus (CaMV) 19S and 35S promoters (U.S. Pat. No. 5,858,642, specifically incorporated herein by reference in its entirety); the figwort mosaic virus promoter (P-FMV, U.S. Pat. No. 6,051,753, specifically incorporated herein by reference in its entirety); actin promoters, such as the rice actin promoter (P-Os.Act 1, U.S. Pat. No. 5,641,876, specifically incorporated herein by reference in its entirety).

Furthermore, the promoters may be altered to contain one or more “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin 1 and rice actin 2 genes. Examples of other enhancers that can be used in accordance with the invention include elements from the CaMV 35S promoter, octopine synthase genes, the maize alcohol dehydrogenase gene, the maize shrunken 1 gene and promoters from non-plant eukaryotes.

Tissue-preferred promoters cause transcription or enhanced transcription of a polynucleotide sequence in specific cells or tissues at specific times during plant development, such as in vegetative or reproductive tissues. Examples of tissue-preferred promoters under developmental control include promoters that initiate transcription primarily in certain tissues, such as vegetative tissues, e.g., roots, leaves or stems, or reproductive tissues, such as fruit, ovules, seeds, pollen, pistils, flowers, or any embryonic tissue, or any combination thereof. Reproductive tissue preferred promoters may be, e.g., ovule-preferred, embryo-preferred, endosperm-preferred, integument-preferred, pollen-preferred, petal-preferred, sepal-preferred, or some combination thereof. Tissue preferred promoter(s) will also include promoters that can cause transcription, or enhanced transcription in a desired plant tissue at a desired plant developmental stage. An example of such a promoter includes, but is not limited to, a seedling or an early seedling preferred promoter. One skilled in the art will recognize that a tissue-preferred promoter may drive expression of operably linked polynucleotide molecules in tissues other than the target tissue. Thus, as used herein, a tissue-preferred promoter is one that drives expression preferentially not only in the target tissue, but may also lead to some expression in other tissues as well.

In one embodiment of this invention, preferential expression in plant green tissues is desired. Promoters of interest for such uses include those from genes such as maize aldolase gene FDA (U.S. patent application publication No. 20040216189, specifically incorporated herein by reference in its entirety), aldolase and pyruvate orthophosphate dikinase (PPDK) (Taniguchi et al. (2000) Plant Cell Physiol. 41(1):42-48).

In another embodiment of this invention, preferential expression in plant root tissue is desired. Exemplary promoter of interest for such uses is derived from Corn Nicotianamine Synthase gene (U.S. patent application publication No. 20030131377, specifically incorporated herein by reference in its entirety) and rice RCC3 promoter (U.S. patent application Ser. No. 11/075,113, specifically incorporated herein by reference in its entirety).

In yet another embodiment of this invention, preferential expression in plant phloem tissue is desired. An exemplary promoter of interest for such use is the rice tungro bacilliform virus (RTBV) promoter (U.S. Pat. No. 5,824,857, specifically incorporated herein by reference in its entirety).

In practicing the present invention, an inducible promoter may also be used to ectopically express the structural gene in the recombinant DNA construct. The inducible promoter may cause conditional expression of a polynucleotide sequence under the influence of changing environmental conditions or developmental conditions. For example, such promoters may cause expression of the polynucleotide sequence at certain temperatures or temperature ranges, or in specific stage(s) of plant development such as in early germination or late maturation stage(s) of a plant. Examples of inducible promoters include, but are not limited to, the light-inducible promoter from the small subunit of ribulose-1,5-bis-phosphate carboxylase (ssRUBISCO); the drought-inducible promoter of maize (Busk et al., Plant J. 11:1285-1295, 1997), the cold, drought, and high salt inducible promoter from potato (Kirch, Plant Mol. Biol. 33:897-909, 1997), and many cold inducible promoters known in the art; for example rd29a and cor15a promoters from Arabidopsis (Genbank ID: D13044 and U01377), blt101 and blt4.8 from barley (Genbank ID: AJ310994 and U63993), wcs120 from wheat (Genbank ID:AF031235), mlip15 from corn (Genbank ID: D26563) and bn115 from Brassica (Genbank ID: U01377).

### Plant Transformation

Various methods for the introduction of a heterologous flavohemoglobin gene encoding, provided by the present invention, into plant cells are available and known to those of skill in the art and include, but are not limited to: (1) physical methods such as microinjection (Capecchi, Cell, 22(2):479-488, 1980), electroporation (Fromm et al., Proc. Natl. Acad. Sci. USA, 82(17):5824-5828, 1985; U.S. Pat. No. 5,384,253) and microprojectile mediated delivery (biolistics or gene gun technology) (Christou et al., Bio/Technology 9:957, 1991; Fynan et al, Proc. Natl. Acad. Sci. USA, 90(24):11478-11482, 1993); (2) virus mediated delivery methods (Clapp, Clin. Perinatol., 20(1):155-168, 1993; Lu et al., J. Exp. Med., 178(6):2089-2096, 1993; Eglitis and Anderson, Biotechniques, 6(7):608-614, 1988; and (3) Agrobacterium-mediated transformation methods.

The most commonly used methods for transformation of plant cells are the Agrobacterium-mediated DNA transfer process (Fraley et al., Proc. Natl. Acad. Sci. U.S.A., 80: 4803, 1983) and the biolistics or microprojectile bombardment mediated process (i.e. the gene gun). Typically, nuclear transformation is desired but where it is desirable to specifically transform plastids, such as chloroplasts or amyloplasts, plant plastids may be transformed utilizing a microprojectile mediated delivery of the desired polynucleotide for certain plant species such as tobacco, Arabidopsis, potato and Brassica species.

Agrobacterium-mediated transformation is achieved through the use of a genetically engineered soil bacterium belonging to the genus Agrobacterium. A disarmed Agrobacterium strain C58 (ABI) harboring a DNA construct can be used for all the experiments. According to this method, the construct is transferred into Agrobacterium by a triparental mating method (Ditta et al., Proc. Natl. Acad. Sci. 77:7347-7351). Liquid cultures of Agrobacterium are initiated from glycerol stocks or from a freshly streaked plate and grown overnight at 26° C.-28° C. with shaking (approximately 150 rpm) to mid-log growth phase in liquid LB medium, pH 7.0 containing 50 mg/1 kanamycin, 50 mg/1 streptomycin and spectinomycin and 25 mg/1 chloramphenicol with 200 μM acetosyringone (AS). The Agrobacterium cells are resuspended in the inoculation medium (liquid CM4C) and the density is adjusted to OD₆₆₀ of 1. Freshly isolated Type II immature HiIIxLH198 and HiII corn embryos are inoculated with Agrobacterium containing a DNA construct of the present invention and co-cultured 2-3 days in the dark at 23° C. The embryos are then transferred to delay media (N6 1-100-12/micro/Carb 500/20 μM AgNO3) and incubated at 28° C. for 4 to 5 days. All subsequent cultures are kept at this temperature. Coleoptiles are removed one week after inoculation. The embryos are transferred to the first selection medium (N61-0-12/Carb 500/0.5 mM glyphosate). Two weeks later, surviving tissues are transferred to the second selection medium (N61-0-12/Carb 500/1.0 mM glyphosate). Surviving callus is sub cultured every 2 weeks until events can be identified. This usually takes 3 subcultures on a desired selection media. Once events are identified, tissue is bulked up for regeneration. For regeneration, callus tissues are transferred to the regeneration medium (MSOD, 0.1 μM ABA) and incubated for two weeks. The regenerating calli are transferred to a high sucrose medium and incubated for two weeks. The plantlets are transferred to MSOD media in a culture vessel and kept for two weeks. Then the plants with roots are transferred into soil. After identifying appropriated transformed plants, plants can be grown to produce desired quantities of seeds of the inventions.

With respect to microprojectile bombardment (U.S. Pat. No. 5,550,318; U.S. Pat. No. 5,538,880; U.S. Pat. No. 5,610,042; and PCT Publication WO 95/06128; each of which is specifically incorporated herein by reference in its entirety), particles are coated with nucleic acids and delivered into cells by a propelling force. An illustrative embodiment of a method for delivering DNA into plant cells by acceleration is the Biolistics Particle Delivery System (BioRad, Hercules, Calif.), which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates.

Microprojectile bombardment techniques are widely applicable, and may be used to transform virtually any plant species. Examples of species that have been transformed by microprojectile bombardment include monocot species such as maize (PCT Publication WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (Van Eck et al. 1995), and legumes in general (U.S. Pat. No. 5,563,055, specifically incorporated herein by reference in its entirety).

For microprojectile bombardment transformation in accordance with the current invention, both physical and biological parameters may be optimized. Physical factors are those that involve manipulating the DNA/microprojectile precipitate or those that affect the flight and velocity of either the macro- or microprojectiles. Biological factors include all steps involved in manipulation of cells before and immediately after bombardment, such as the osmotic adjustment of target cells to help alleviate the trauma associated with bombardment, the orientation of an immature embryo or other target tissue relative to the particle trajectory, and also the nature of the transforming DNA, such as linearized DNA or intact supercoiled plasmids. It is believed that pre-bombardment manipulations are especially important for successful transformation of immature embryos.

Accordingly, it is contemplated that one may wish to adjust various of the bombardment parameters in small scale studies to fully optimize the conditions. One may particularly wish to adjust physical parameters such as DNA concentration, gap distance, flight distance, tissue distance, and helium pressure. It further is contemplated that the grade of helium may effect transformation efficiency. One also may optimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation.

To select or score for transformed plant cells regardless of transformation methodology, the DNA introduced into the cell contains a gene that functions in a regenerable plant tissue to produce a compound that confers upon the plant tissue resistance to an otherwise toxic compound. Genes of interest for use as a selectable, screenable, or scorable marker will include but are not limited to GUS, green fluorescent protein (GFP), luciferase (LUX), antibiotic or herbicide tolerance genes. Examples of antibiotic resistance genes include the penicillins, kanamycin (and neomycin, G418, bleomycin); methotrexate (and trimethoprim); chloramphenicol; kanamycin and tetracycline.

Particularly preferred selectable marker genes for use in the present invention will include genes that confer resistance to compounds such as antibiotics like kanamycin (nptII), hygromycin B (aph IV) and gentamycin (aac3 and aacC4) (Dekeyser et al., Plant Physiol., 90:217-223, 1989), and herbicides like glyphosate (Della-Cioppa et al., Bio/Technology, 5:579-584, 1987). Other selection devices can also be implemented including but not limited to tolerance to phosphinothricin, bialaphos, and positive selection mechanisms (Joersbo et al., Mol. Breed., 4:111-117, 1998) and are considered within the scope of the present invention. The regeneration, development, and cultivation of plants from various transformed explants are well documented in the art. This regeneration and growth process typically includes the steps of selecting transformed cells and culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Cells that survive the exposure to the selective agent, or cells that have been scored positive in a screening assay, may be cultured in media that supports regeneration of plants. In an embodiment, MS and N6 media may be modified by including further substances such as growth regulators. A preferred growth regulator for such purposes is dicamba or 2,4-D. However, other growth regulators may be employed, including NAA, NAA+2,4-D or perhaps even picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 weeks, then transferred to media conducive to maturation of embryoids. Cultures are transferred every 2 weeks on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soilless plant growth mix, and hardened off, e.g., in an environmentally controlled chamber at about 85% relative humidity, 600 ppm CO₂, and 25-250 microeinsteins m⁻² s⁻¹ of light, prior to transfer to a greenhouse or growth chamber for maturation. Plants are preferably matured either in a growth chamber or greenhouse. Plants are regenerated from about 6 wk to 10 months after a transformant is identified, depending on the initial tissue. During regeneration, cells are grown on solid media in tissue culture vessels. Illustrative embodiments of such vessels are petri dishes and Plant Cons. Regenerating plants are preferably grown at about 19 to 28° C. After the regenerating plants have reached the stage of shoot and root development, they may be transferred to a greenhouse for further growth and testing.

Note, however, that seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/1 agarose. In embryo rescue, large embryos (defined as greater than 3 mm in length) are germinated directly on an appropriate media. Embryos smaller than that may be cultured for 1 wk on media containing the above ingredients along with 10⁻⁵M abscisic acid and then transferred to growth regulator-free medium for germination.

The present invention can be used with any transformable cell or tissue. By transformable as used herein is meant a cell or tissue that is capable of further propagation to give rise to a plant. Those of skill in the art recognize that a number of plant cells or tissues are transformable in which after insertion of exogenous DNA and appropriate culture conditions the plant cells or tissues can form into a differentiated plant. Tissue suitable for these purposes can include but is not limited to immature embryos, scutellar tissue, suspension cell cultures, immature inflorescence, shoot meristem, nodal explants, callus tissue, hypocotyl tissue, cotyledons, roots, and leaves.

Any suitable plant culture medium can be used. Examples of suitable media will include but are not limited to MS-based media (Murashige and Skoog, Physiol. Plant, 15:473-497, 1962) or N6-based media (Chu et al., Scientia Sinica 18:659, 1975) supplemented with additional plant growth regulators including but not limited to auxins such as picloram (4-amino-3,5,6-trichloropicolinic acid), 2,4-D (2,4-dichlorophenoxyacetic acid) and dicamba (3,6-dichloroanisic acid); cytokinins such as BAP (6-benzylaminopurine) and kinetin; ABA; and gibberellins. Other media additives can include but are not limited to amino acids, macroelements, iron, microelements, vitamins and organics, carbohydrates, undefined media components such as casein hydrolysates, with or without an appropriate gelling agent such as a form of agar, such as a low melting point agarose or Gelrite if desired. Those of skill in the art are familiar with the variety of tissue culture media, which when supplemented appropriately, support plant tissue growth and development and are suitable for plant transformation and regeneration. These tissue culture media can either be purchased as a commercial preparation, or custom prepared and modified. Examples of such media will include but are not limited to Murashige and Skoog (Murashige and Skoog, Physiol. Plant, 15:473-497, 1962), N6 (Chu et al., Scientia Sinica 18:659, 1975), Linsmaier and Skoog (Linsmaier and Skoog, Physio. Plant., 18: 100, 1965), Uchimiya and Murashige (Uchimiya and Murashige, Plant Physiol. 15:473, 1962), Gamborg's B5 media (Gamborg et al., Exp. Cell Res., 50:151, 1968), D medium (Duncan et al., Planta, 165:322-332, 1985), McCown's Woody plant media (McCown and Lloyd, HortScience 16:453, 1981), Nitsch and Nitsch (Nitsch and Nitsch, Science 163:85-87, 1969), and Schenk and Hildebrandt (Schenk and Hildebrandt, Can. J. Bot. 50:199-204, 1972) or derivations of these media supplemented accordingly. Those of skill in the art are aware that media and media supplements such as nutrients and growth regulators for use in transformation and regeneration and other culture conditions such as light intensity during incubation, pH, and incubation temperatures that can be optimized for the particular variety of interest.

Transgenic Plants Expressing a Heterologous Flavohemoglobin Protein have Improved Agronomic Trait(s)

In one embodiment of the present invention, transgenic plants expressing E. coli HMP, have been generated and have been shown to contain a higher level of chlorophyll content, under a limiting nitrogen growth condition, as compared to control plants. The higher level of chlorophyll content is a characteristics of more robust growth. In another aspect, according to the present invention, the transgenic plants expressing E. coli HMP also exhibit more robust growth under a sufficient nitrogen growth condition, shown as increased shoot fresh mass. In yet another aspect, according to the present invention, expressing E. coli HMP in corn plants significantly reduces the level of NO in leaf tissues. In still another aspect, according to the present invention, transgenic corn plants expressing E. coli HMP also have shown to have increased seed yield under field conditions.

In another embodiment of the present invention, transgenic corn plants expressing yeast YHB1 also have been generated and have been shown to have an increased yield.

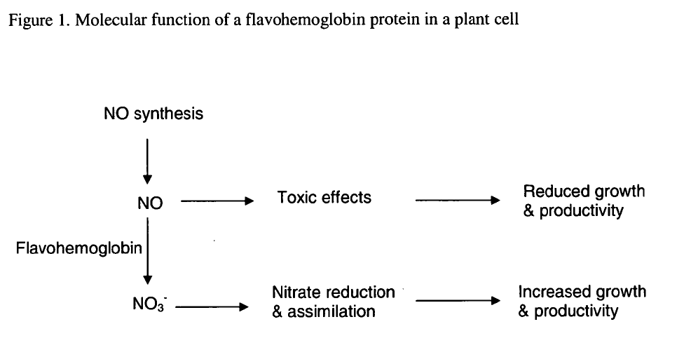

As illustrated in FIG. 1, in accordance to the present invention, we contemplate that, under the limiting nitrogen growth condition, the presence of flavohemoglobin may enhance plant growth by increasing available nitrate, whereas, under the sufficient nitrogen growth condition or limiting nitrogen condition, the presence of flavohemoglobin may enhance plant growth by reducing toxic effect of NO.

Also in accordance of the present invention, transgenic plants expressing a heterologous flavohemoglobin having an amino acid sequence selected from the group consisting of SEQ ID NO: 130 through SEQ ID NO: 256, which are identified as the homologs of the E. coli HMP protein by the present invention.

Plants of the present invention include, but not limited to, Acacia, alfalfa, aneth, apple, apricot, artichoke, arugula, asparagus, avocado, banana, barley, beans, beet, blackberry, blueberry, broccoli, brussels sprouts, cabbage, canola, cantaloupe, carrot, cassava, cauliflower, celery, cherry, cilantro, citrus, clementine, coffee, corn, cotton, cucumber, Douglas fir, eggplant, endive, escarole, eucalyptus, fennel, figs, forest trees, gourd, grape, grapefruit, honey dew, jicama, kiwifruit, lettuce, leeks, lemon, lime, loblolly pine, mango, melon, mushroom, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, tangerine, tea, tobacco, tomato, turf, a vine, watermelon, wheat, yams, and zucchini. Crop plants are defined as plants, which are cultivated to produce one or more commercial product. Examples of such crops or crop plants include but are not limited to soybean, canola, rape, cotton (cottonseeds), sunflower, and grains such as corn, wheat, rice, and rye. Rape, rapeseed or canola is used synonymously in the present disclosure.

The transgenic plants of the present invention may be productively cultivated under limiting nitrogen growth conditions (i.e., nitrogen-poor soils and low nitrogen fertilizer inputs) that would cause the growth of wild-type plants to cease, to be so diminished as to make the wild-type plants practically useless, or cause a significant yield reduction of wild-type plants. The transgenic plants also may be advantageously used to achieve earlier maturing, faster growing, and/or higher yielding crops and/or produce more nutritious foods and animal feedstocks when cultivated using sufficient nitrogen growth conditions (i.e., soils or media containing or receiving sufficient amounts of nitrogen nutrients to sustain healthy plant growth). In another aspect, transgenic plants with increased nitrogen use efficiency provided by the present invention will have environmental benefits in general, such as reducing the amount of nitrate leashed from soil and into ground water.

The following examples are provided to better elucidate the practice of the present invention and should not be interpreted in any way to limit the scope of the present invention. Those skilled in the art will recognize that various modifications, additions, substitutions, truncations, etc., can be made to the methods and genes described herein while not departing from the spirit and scope of the present invention.

### EXAMPLES

### Example 1

### Construct for Plant Transformation

### A. Corn Transformation Constructs

GATEWAY™ destination vectors (available from Invitrogen Life Technologies, Carlsbad, Calif.) can be constructed for each DNA molecule disclosed herein for corn transformation. The elements of each destination vector are summarized in Table 2 below and include a selectable marker transcription region and a DNA insertion transcription region. The selectable marker transcription region comprises a Cauliflower Mosaic Virus 35S promoter operably linked to a gene encoding neomycin phosphotransferase II (nptII) followed by both the 3′ region of the Agrobacterium tumefaciens nopaline synthase gene (nos) and the 3′ region of the potato proteinase inhibitor II (pinII) gene. The DNA insertion transcription region comprises a rice actin 1 promoter, a rice actin 1 exon 1 intron1 enhancer, an att-flanked insertion site and the 3′ region of the potato pinII gene. Following standard procedures provided by Invitrogen the att-flanked insertion region is replaced by recombination with trait-improving DNA, in a sense orientation for expression of a flavohemoglobin protein. Although the vector with the flavohemoglobin gene disclosed herein inserted at the att-flanked insertion region is useful for plant transformation by direct DNA delivery, such as microprojectile bombardment, it is preferable to bombard target plant tissue with tandem transcription units that have been cut from the vector.

TABLE 2
Elements of an exemplary corn transformation vector

| FUNCTION | ELEMENT | REFERENCE |
|---|---|---|
| DNA insertion | Rice actin 1 promoter | U.S. Pat. No. 5,641,876 |
| transcription region | Rice actin_1_exon_1_intron_1 | U.S. Pat. No. 5,641,876 |
| | enhancer | |
| DNA insertion | AttR1 | GATEWAY ™Cloning Technology |
| transcription region | | Instruction Manual |
| (att - flanked insertin | CmR gene | GATEWAY ™Cloning Technology |
| region) | | Instruction Manual |
| | ccdA, ccdB genes | GATEWAY ™Cloning Technology |
| | | Instruction Manual |
| | attR2 | GATEWAY ™Cloning Technology |
| | | Instruction Manual |
| DNA insertion | Potato pinII 3′ region | An, et al., (1989) Plant Cell 1: 115-122 |
| transcription region | | |
| selectable marker | CaMV 35S promoter | U.S. Pat. No. 5,858,742 |
| transcription region | nptII selectable marker | U.S. Pat. No. 5,858,742 |
| | nos 3region | U.S. Pat. No. 5,858,742 |
| | PinII 3′ region | An, et al., (1989) Plant Cell 1: 115-122 |
| E. coli maintenance | ColE1 origin of | / |
| region | replication | |
| | F1 origin of replication | / |
| | Bla ampicillin resistance | / |

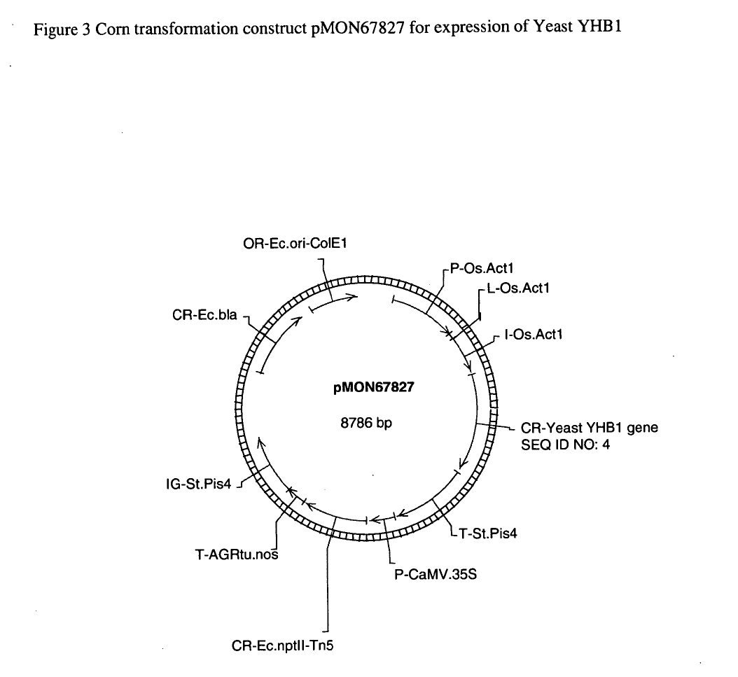

Exemplary such corn transformation constructs made by the present invention include pMON69471 comprising SEQ NO:3 as shown in FIG. 2, pMON67827 comprising SEQ NO: 4 as shown in FIG. 3.

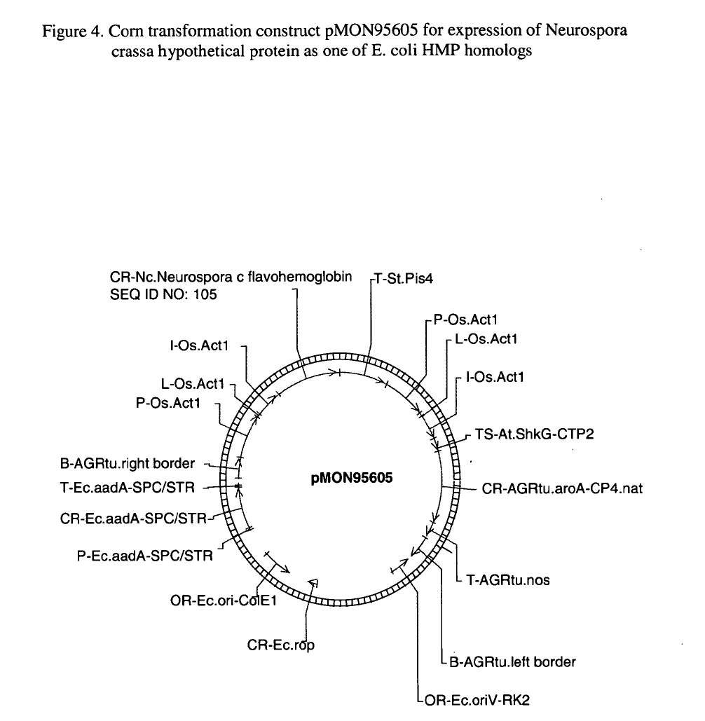

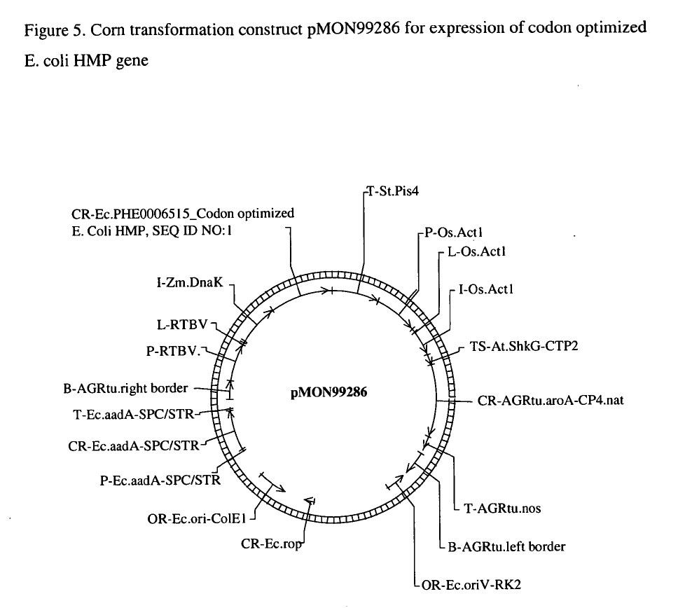

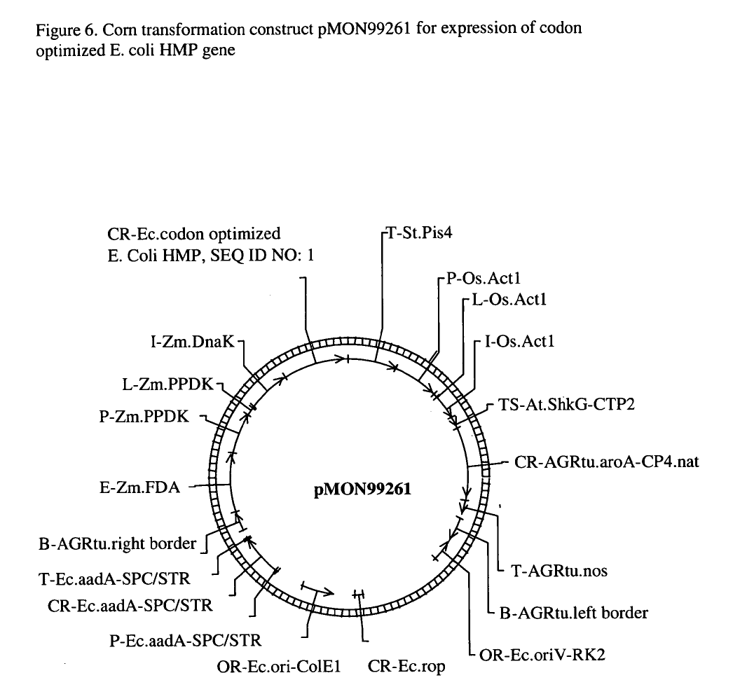

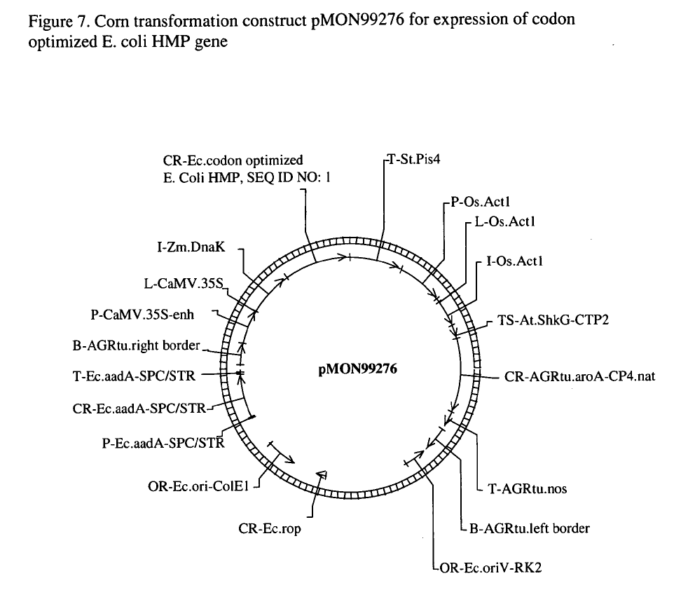

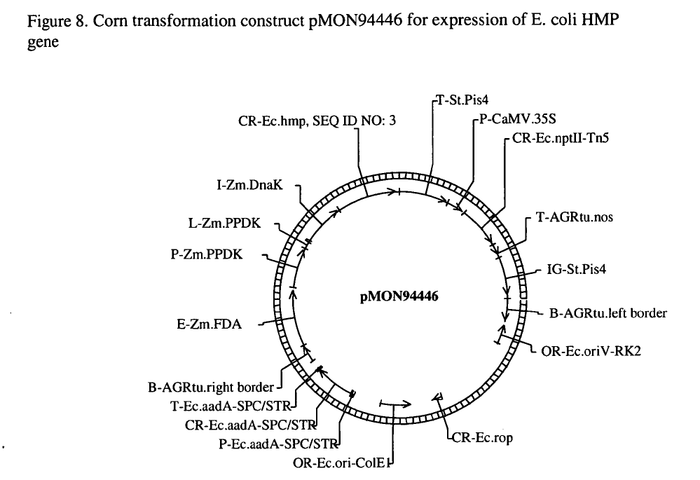

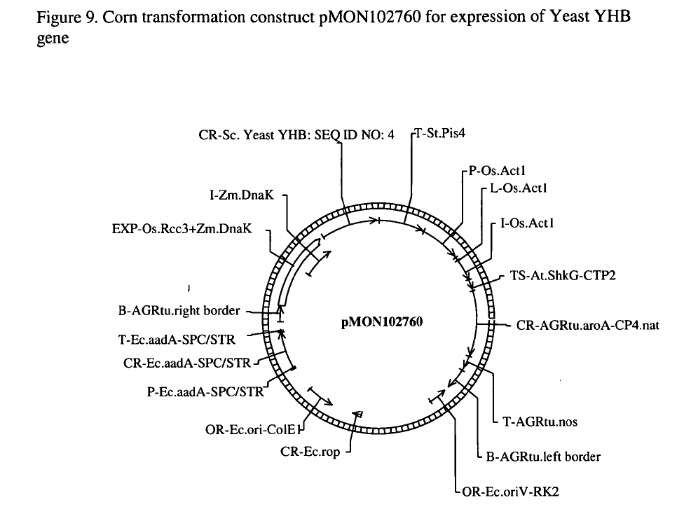

For Agrobacterium-mediated transformation of plants the vector also comprises T-DNA borders from Agrobacterium flanking the transcription units. Elements of an exemplary expression vector, pMON95605, are illustrated in FIG. 4 and Table 3. Elements of another exemplary expression vector, pMON99286, are illustrated in FIG. 5 and Table 4. Yet elements of another exemplary expression vector, pMON99261, are illustrated in FIG. 6 and Table 5. Yet elements of another exemplary expression vector, pMON99276, are illustrated in FIG. 7 and Table 6. Yet elements of another exemplary expression vector, pMON94446, are illustrated in FIG. 8 and Table 7. Elements of another exemplary expression vector, pMON102760, are illustrated in FIG. 9 and Table 8. These corn transformation constructs were assembled using the technology known in the art.

TABLE 3
Annotation of element names used in plasmid map of pMON96505

| Element | |
|---|---|
| name in FIGURES | Annotation |
| CR-AGRtu.aroA- | Coding region for native bacterial strain CP4 aroA |
| CP4.nat | gene, encoding class II EPSPS enzyme |
| CR-Ec.aadA- | Coding region for Tn7 adenylyltransferase |
| SPC/STR | (AAD(3″)) conferring spectinomycin and |
| | streptomycin resistance |
| CR-Ec.bla | The coding sequence for beta-lactamase derived |
| CR-Ec.nptII-Tn5 | coding region for nptII from E. coli |
| CR-Ec.rop | Coding region for repressor of primer from the |
| | ColE1 plasmid. Also known as rom. Expression of |
| | this gene product interferes with primer binding at |
| | the origin of replication, keeping plasmid copy |
| | number low. |
| IG-St.Pis4 | Intergenic region of the potato proteinase inhibitor |
| | II gene |
| I-Os-Act1 | First intron and flanking UTR exon sequences from |
| | the rice actin 1 gene |
| L-Os.Act1 | Leader (first exon) from the rice actin 1 gene |
| OR-Ec.ori-ColE1 | Minimal origin of replication from the Escherichia |
| | coli plasmid, ColE1 |
| OR-Ec.oriV-RK2 | Vegetative origin of replication used by |
| | Agrobacterium tumefaciens |
| P-CaMv.35S | promoter and 5′UTR for the CaMV 35S RNA |
| P-Ec.aadA-SPC/ | promoter of aadA for spectinomycin and |
| STR | streptomycin resistance gene expression |
| p-Os.Act1 | Promoter from the rice actin gene |
| T-AGRtu.nos | transcription termination sequence of from nopaline |
| | synthase gene from Agrobacterium |
| T-Ec.aadA-SPC/ | terminator of aadA for spectinomycin and |
| STR | streptomycin resistance gene expression |
| TS-At.ShkG-CTP2 | Transit peptide from Arabidopsis EPSPS-CTP2 |
| | gene |
| T-St.Pis4 | The 3′ non-translated region of the potato |
| | proteinase inhibitor II gene which functions to |
| | direct polyadenylation of the mRNA |
| B-AGRtu.left border | Left border sequence for T-DNA transfer |
| B-AGRtu.right | right border sequence for T-DNA transfer |
| border | |

TABLE 4
Annotation of element names used in plasmid map of pMON99286

| Element Name | Coordinates | Annotation |
|---|---|---|
| T-St.Pis4 | 19-961 | The 3′ non-translated region of the potato |
| | | proteinase inhibitor II gene which functions |
| | | to direct polyadenylation of the mRNA |
| P-Os.Act1 | 977-1817 | Promoter from the rice actin 1 gene |
| L-Os.Act1 | 1818-1897 | Leader (first exon) from the rice actin 1 gene |
| I-Os.Act1 | 1898-2375 | First intron and flanking UTR exon |
| | | sequences from the rice actin 1 gene |
| TS-At.ShkG-CTP2 | 2385-2612 | Transit peptide from Arabidopsis EPSPS- |
| | | CTP2 gene |
| CR-AGRtu.aroA- | 2613-3980 | Coding region for native bacterial strain CP4 |
| CP4.nat | | aroA gene, encoding class II EPSPS enzyme |
| T-AGRtu.nos | 3996-4248 | transcription termination sequence of from |
| | | nopaline synthase gene from Agrobacterium |
| B-AGRtu.left border | 4377-4818 | Left border sequence for T-DNA transfer |
| OR-Ec.oriV-RK2 | 4875-5271 | Vegetative origin of replication used by |
| | | Agrobacterium tumefaciens |
| CR-Ec.rop | 6780-6971 | Coding region for repressor of primer from |
| | | the ColE1 plasmid. Also known as rom. |
| | | Expression of this gene product interferes |
| | | with primer binding at the origin of |
| | | replication, keeping plasmid copy number |
| | | low. |
| OR-Ec.ori-ColE1 | 7399-7987 | Minimal origin of replication from the |
| | | Escherichia coli plasmid, ColE1 |
| P-Ec.aadA-SPC/STR | 8518-8559 | promoter of aadA for spectinomycin and |
| | | streptomycin resistance gene expression |
| CR-Ec.aadA-SPC/STR | 8560-9348 | Coding region for Tn7 adenylyltransferase |
| | | (AAD(3″)) conferring spectinomycin and |
| | | streptomycin resistance |
| T-Ec.aadA-SPC/STR | 9349-9406 | terminator of aadA for spectinomycin and |
| | | streptomycin resistance gene expression |
| B-AGRtu.right border | 9543-9899 | Right border sequence for T-DNA transfer |
| P-RTBV | 9925-10650 | Promoter from rice tungro bacilliform virus. |
| L-RTBV | 10651-10690 | 5′ untranslated region from the rice tungro |
| | | bacilliform virus full length transcript. |
| I-Zm.DnaK | 10711-11514 | Zea mays HSP70 intron with flanking exon |
| | | sequence enhances expression in plants. |
| CR- | 11551-12741 | SEQ ID NO: 1 |
| Ec.PHE0006515_Codon- | | |
| optimized E. coli HMP | | |

TABLE 5
Annotation of element names used in plasmid map of pMON99261

| Element Name | Coordinates | Annotation |
|---|---|---|
| T-St.Pis4 | 19-961 | The 3′ non-translated region of the potato |
| | | proteinase inhibitor II gene which functions to |
| | | direct polyadenylation of the mRNA |
| P-Os.Act1 | 977-1817 | Promoter from the rice actin 1 gene |
| L-Os.Act1 | 1818-1897 | Leader (first exon) from the rice actin 1 gene |
| I-Os.Act1 | 1898-2375 | First intron and flanking UTR exon sequences |
| | | from the rice actin 1 gene |
| TS-At.ShkG-CTP2 | 2385-2612 | Transit peptide from Arabidopsis EPSPS- |
| | | CTP2 gene |
| CR-AGRtu.aroA-CP4.nat | 2613-3980 | Coding region for native bacterial strain CP4 |
| | | aroA gene, encoding class II EPSPS enzyme |
| T-AGRtu.nos | 3996-4248 | transcription termination sequence of from |
| | | nopaline synthase gene from Agrobacterium |
| B-AGRtu.left border | 4347-4788 | Left border sequence for T-DNA transfer |
| OR-Ec.oriV-RK2 | 4875-5271 | Vegetative origin of replication used by |
| | | Agrobacterium tumefaciens |
| CR-Ec.rop | 6780-6971 | Coding region for repressor of primer from |
| | | the ColE1 plasmid. Also known as rom. |
| | | Expression of this gene product interferes |
| | | with primer binding at the origin of |
| | | replication, keeping plasmid copy number |
| | | low. |
| OR-Ec.ori-ColE1 | 7399-7987 | Minimal origin of replication from the |
| | | Escherichia coli plasmid, ColE1 |
| P-Ec.aadA-SPC/STR | 8518-8559 | promoter of aadA for spectinomycin and |
| | | streptomycin resistance gene expression |
| CR-Ec.aadA-SPC/STR | 8560-9348 | Coding region for Tn7 adenylyltransferase |
| | | (AAD(3″)) conferring spectinomycin and |
| | | streptomycin resistance |
| T-Ec.aadA-SPC/STR | 9349-9406 | terminator of aadA for spectinomycin and |
| | | streptomycin resistance gene expression |
| B-AGRtu.right border | 9543-9899 | Right border sequence for T-DNA transfer |
| E-Zm.FDA | 9922-11036 | enhancer derived from the promoter region of |
| | | corn fructose-bisphosphate aldolase |
| P-Zm.PPDK-1:1:10 | 11078-11863 | Promoter from corn pyruvate orthophosphate |
| | | dikinase gene |
| L-Zm.PPDK | 11864-12028 | 5′ untranslated region from corn pyruvate |
| | | orthophosphate dikinase gene |
| I-Zm.DnaK | 12042-12845 | Zea mays HSP70 intron with flanking exon |
| | | sequence enhances expression in plants |
| CR- | 12882-14072 | SEQ ID NO: 1 |
| Ec.PHE0006515_Codon- | | |
| optimized E. coli HMP | | |

TABLE 6
Annotation of element names used in plasmid map of pMON99276

| Element Name | Coordinates | Annotation |
|---|---|---|
| T-St.Pis4 | 19-961 | The 3′ non-translated region of the potato |
| | | proteinase inhibitor II gene which functions to |
| | | direct polyadenylation of the mRNA |
| P-Os.Act1 | 1007-1847 | Promoter from the rice actin 1 gene |
| L-Os.Act1 | 1848-1927 | Leader (first exon) from the rice actin 1 gene |
| I-Os.Act1 | 1928-2405 | First intron and flanking UTR exon sequences |
| | | from the rice actin 1 gene |
| TS-At.ShkG-CTP2 | 2415-2642 | Transit peptide from Arabidopsis EPSPS- |
| | | CTP2 gene |
| CR-AGRtu.aroA-CP4.nat | 2643-4010 | Coding region for native bacterial strain CP4 |
| | | aroA gene, encoding class II EPSPS enzyme |
| T-AGRtu.nos | 4026-4278 | transcription termination sequence of from |
| | | nopaline synthase gene from Agrobacterium |
| B-AGRtu.left border | 4377-4818 | Left border sequence for T-DNA transfer |
| OR-Ec.oriV-RK2 | 4905-5301 | Vegetative origin of replication used by |
| | | Agrobacterium tumefaciens |
| CR-Ec.rop | 6810-7001 | Coding region for repressor of primer from |
| | | the ColE1 plasmid. Also known as rom. |
| | | Expression of this gene product interferes |
| | | with primer binding at the origin of |
| | | replication, keeping plasmid copy number |
| | | low. |
| OR-Ec.ori-ColE1 | 7429-8017 | Minimal origin of replication from the |
| | | Escherichia coli plasmid, ColE1 |
| P-Ec.aadA-SPC/STR | 8548-8589 | promoter of aadA for spectinomycin and |
| | | streptomycin resistance gene expression |
| CR-Ec.aadA-SPC/STR | 8590-9378 | Coding region for Tn7 adenylyltransferase |
| | | (AAD(3″)) conferring spectinomycin and |
| | | streptomycin resistance |
| T-Ec.aadA-SPC/STR | 9379-9436 | terminator of aadA for spectinomycin and |
| | | streptomycin resistance gene expression |
| B-AGRtu.right border | 9573-9929 | Right border sequence for T-DNA transfer |
| P-CaMV.35S | 9956-10567 | Promoter for 35S RNA from CaMV |
| | | containing a duplication of the −90 to −350 |
| | | region. |
| L-CaMV.35S | 10568-10576 | 5′ UTR from the 35S RNA of CaMV. |
| I-Zm.DnaK | 10583-11386 | Zea mays HSP70 intron with flanking exon |
| | | sequence enhances expression in plant |
| CR- | 11423-12613 | SEQ ID NO: 1 |
| Ec.PHE0006515_Codon- | | |
| optimized E. coli HMP | | |

TABLE 7
Annotation of element names used in plasmid map of pMON94446

| Element Name | Coordinates | Annotation |
|---|---|---|
| P-CaMV.35S | 1011-1303 | Promoter for the 35S RNA from CaMV |
| CR-Ec.nptII- | 1368-2175 | Confers resistance to neomycin and kanamycin |
| Tn5 | | |
| T-AGRtu.nos | 2204-2456 | transcription termination sequence of from nopaline |
| | | synthase gene from Agrobacterium |
| IG-St.Pis4 | 2468-3214 | Intergenic region of the potato proteinase inhibitor II gene |
| B-AGRtu.left | 3277-3718 | Left border sequence for T-DNA transfer |
| border | | |
| OR-Ec.oriV- | 3805-4201 | Vegetative origin of replication used by |
| RK2 | | Agrobacterium tumefaciens |
| CR-Ec.rop | 5710-5901 | Coding region for repressor of primer from the ColE1 |
| | | plasmid. Also known as rom. Expression of this gene |
| | | product interferes with primer binding at the origin of |
| | | replication, keeping plasmid copy number low. |
| OR-Ec.ori- | 6329-6917 | Minimal origin of replication from the Escherichia coli |
| ColE1 | | plasmid, ColE1 |
| P-Ec.aadA- | 7448-7489 | promoter of aadA for spectinomycin and streptomycin |
| SPC/STR | | resistance gene expression |
| CR-Ec.aadA- | 7490-8278 | Coding region for Tn7 adenylyltransferase (AAD(3″)) |
| SPC/STR | | conferring spectinomycin and streptomycin resistance |
| T-Ec.aadA- | 8279-8336 | terminator of aadA for spectinomycin and |
| SPC/STR | | streptomycin resistance gene expression |
| B-AGRtu.right | 8473-8829 | Right border sequence for T-DNA transfer |
| border | | |
| E-Zm.FDA | 8852-9966 | enhancer derived from the promoter region of corn |
| | | fructose-bisphosphate aldolase. |
| P-Zm.PPDK | 10008-10793 | Promoter from corn pyruvate orthophosphate dikinase |
| | | gene |
| L-Zm.PPDK | 10794-10958 | 5′ untranslated region from corn pyruvate |
| | | orthophosphate dikinase gene |
| I-Zm.DnaK | 10972-11775 | Zea mays HSP70 intron with flanking exon sequence |
| | | enhances expression in plants |
| CR-Ec.hmp | 11812-13002 | coding region of E. coli HMP gene |
| T-St.Pis4 | 24-966 | The 3′ non-translated region of the potato proteinase |
| | | inhibitor II gene which functions to direct |
| | | polyadenylation of the mRNA |

TABLE 8
Annotation of element names used in plasmid map of pMON102760

| Element Name | Coordinates | Annotation |
|---|---|---|
| P-Os.Act1 | 1025-1865 | Promoter from the rice actin 1 gene |
| L-Os.Act1 | 1866-1945 | Leader (first exon) from the rice actin 1 gene |
| I-Os.Act1 | 1946-2423 | First intron and flanking UTR exon sequences |
| | | from the rice actin 1 gene |
| TS-At.ShkG-CTP2 | 2433-2660 | Transit peptide from Arabidopsis EPSPS-CTP2 gene |
| CR-AGRtu.aroA- | 2661-4028 | Coding region for native bacterial strain CP4 |
| CP4.nat | | aroA gene, encoding class II EPSPS enzyme |
| T-AGRtu.nos | 4044-4296 | transcription termination sequence of from |
| | | nopaline synthase gene from Agrobacterium |
| B-AGRtu.left border | 4395-4836 | Left border sequence for T-DNA transfer |
| OR-Ec.oriV-RK2 | 4923-5319 | Vegetative origin of replication used by |
| | | Agrobacterium tumefaciens |
| CR-Ec.rop | 6828-7019 | Coding region for repressor of primer from the |
| | | ColE1 plasmid. Also known as rom. |
| | | Expression of this gene product interferes with |
| | | primer binding at the origin of replication, |
| | | keeping plasmid copy number low. |
| OR-Ec.ori-ColE1 | 7447-8035 | Minimal origin of replication from the |
| | | Escherichia coli plasmid, ColE1 |
| P-Ec.aadA-SPC/STR | 8566-8607 | promoter of aadA for spectinomycin and |
| | | streptomycin resistance gene expression |
| CR-Ec.aadA-SPC/STR | 8608-9396 | Coding region for Tn7 adenylyltransferase |
| | | (AAD(3″)) conferring spectinomycin and |
| | | streptomycin resistance |
| T-Ec.aadA-SPC/STR | 9397-9454 | terminator of aadA for spectinomycin and |
| | | streptomycin resistance gene expression |
| B-AGRtu.right border | 9591-9947 | Right border sequence for T-DNA transfer |
| EXP- | 9969-11635 | Promoter and 5′ untranslated region from a rice |
| Os.Rcc3 + Zm.DnaK | | root gene plus the corn hsp70 intron |
| P-Os.Rcc3 | 9969-10726 | / |
| L-Os.Rcc3 | 10727-10825 | / |
| I-Zm.DnaK | 10832-11635 | / |
| CR-Sc.yeast | 11672-12871 | coding region of yeast flabohemoglobin gene |
| flavohemoglobin | | |
| T-St.Pis4 | 37-979 | The 3′ non-translated region of the potato |
| | | proteinase inhibitor II gene which functions to |
| | | direct polyadenylation of the mRNA |

Constructs for Agrobacterium-mediated transformation are prepared with each of the flavohemoglobin genes with the DNA solely in sense orientation for expression of the cognate flavohemoglobin protein.

Each construct is transformed into corn callus which is propagated into a plant that is grown to produce transgenic seed. Progeny plants are self-pollinated to produce seed which is selected for homozygous seed. Homozygous seed is used for producing inbred plants, for introgressing the trait into elite lines, and for crossing to make hybrid seed. Transgenic corn including inbred and hybrids are also produced with DNA from each of the identified homologs.

### B. Soybean Transformation Construct

Constructs for use in transformation of soybean may be prepared by restriction enzyme based cloning into a common expression vector. Elements of an exemplary common expression vector are shown in Table 9 below and include a selectable marker expression cassette and a gene of interest expression cassette. The selectable marker expression cassette comprises Arabidopsis act 7 gene (AtAct7) promoter with intron and 5′UTR, the transit peptide of Arabidopsis EPSPS, the synthetic CP4 coding region with dicot preferred codon usage and a 3′ UTR of the nopaline synthase gene. The gene of interest expression cassette comprises a Cauliflower Mosaic Virus 35S promoter operably linked to a trait-improving gene in a sense orientation for expression of a flavohemoglogin.

Vectors similar to that described above may be constructed for use in Agrobacterium mediated soybean transformation systems, with each of the flavohemoglobin genes selected from the group consisting of SEQ ID NO: 1 though SEQ ID NO: 4, and SEQ ID NO: 7 through SEQ ID NO: 129, and SEQ ID NO: 260 with the DNA in sense orientation for expression of the cognate protein. Transgenic soybean plants expressing a heterologous flavohemoglobin protein are produced. Transgenic soybean plants are also produced with DNA from each of the identified homologs and provide seeds for plants with improved agronomic traits.

TABLE 9
Elements of an exemplary soybean transformation construct

| Function | Element | Reference |
|---|---|---|
| Agro transformation | B-ARGtu.right border | Depicker, A., et al., |
| | | (1982) Mol Appl Genet |
| | | 1: 561-573 |
| Antibiotic resistance | CR-Ec.aadA-SPC/STR | / |
| Repressor of primers from the ColE1 | CR-Ec.rop | / |
| plasmid | | |
| Origin of replication | OR-Ec.oriV-RK2 | / |
| Agro transformation | B-ARGtu.left border | Barker, R. F., et al., (1983) |
| | | Plant Mol Biol 2: 335-350 |
| Plant selectable marker expression | Arabidopsis act 7 gene | McDowell, et al., (1996) |
| cassette | (AtAct7) promoter with | Plant Physiol. 111: 699-711. |
| | intron and 5′UTR | |
| | 5′ UTR of Arabidopsis act 7 | |
| | gene | |
| | Intron in 5′UTR of AtAct7 | |
| | Transit peptide region of | Klee, H. J., et al., (1987) |
| | Arabidopsis EPSPS | MGG 210: 437-442 |
| | Synthetic CP4 coding region | / |
| | with dicot preferred codon | |
| | usage | |
| | A 3′ UTR of the nopaline | U.S. Pat. No. 5,858,742 |
| | synthase gene of | |
| | Agrobacterium tumefaciens | |
| | Ti plasmid | |
| Plant gene of interest expression | Promoter for 35S RNA from | U.S. Pat. No. 5,322,938 |
| cassette | CaMV containing a | |
| | duplication of the −90 to −350 | |
| | region | |
| | Gene of interest insertion site | / |
| | Cotton E6 3′ end | GenBank accession |
| | | U30508 |

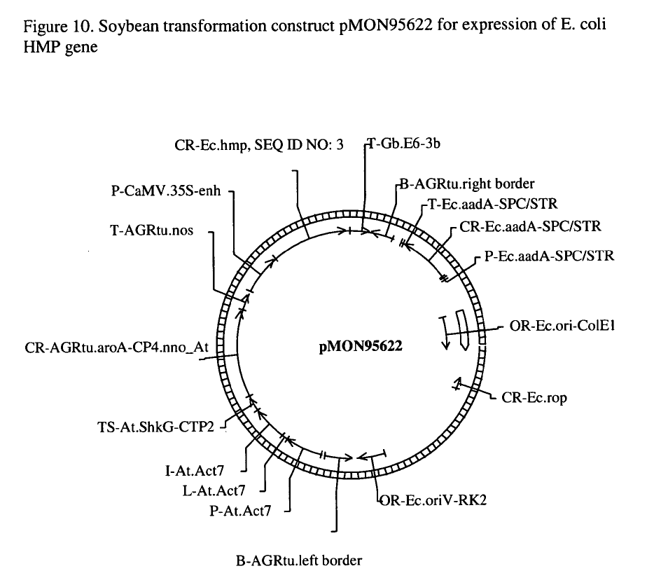

Exemplary such soybean transformation constructs made by the present invention include pMON95622 comprising SEQ NO:3 as shown in FIG. 10.

### Example 2

### Characterization of Transgene Expression

The constructs, pMON69471 was constructed with a sequence derived from the 3′ region of the potato pinII gene, which could be used to assay the relative level of transgene expression. The total RNA was extracted from the tissue lysates by regular methods known in the art and the extracted mRNA was analyzed by Taqman® with probes specific to the potato protease inhibitor (PINII) terminator. Values represent the mean from four individual plants.

The primers for PINII terminator amplification are the followings: PinII F-4 (forward primer) GATGCACACATAGTGACATGCTAATCAC (SEQ ID NO: 267), PinII Probe 4 ATTACACATAACACACAACTTTGATGCCCACAT (SEQ ID NO: 268), PinII R-4 (reverse primer) GGATGATCTCTTTCTCTTATTCAGATAATTAG (SEQ ID NO: 269). Within each PCR reaction, a standard RNA 18S rRNA amplification was used as an internal control. The primers for 18S rRNA amplification are the followings: the forward primer CGTCCCTGCCCTTTGTACAC (SEQ ID NO: 270), the reverse primer CGAACACTTCACCGGATCATT (SEQ ID NO: 271) and the internal primer vic-CCGCCCGTCGCTCCTACCGAT-tamra (SEQ ID NO: 272). The RT-PCR conditions were 48° C. for 30 min, 95° C. for 10 min, 95° C. for 15 sec, and 56° C. for 1 min for 40 cycles.

TABLE 10
Relative transgene expression levels in
transgenic plants comprising SEQ NO: 3

| Transgenic | |
|---|---|
| Event ID | PinII Expression |
| Wild-type | 1 |
| ZM_M20388 | 689 |
| ZM_M21505 | 274 |
| ZM_M21509 | 319 |
| ZM_M21516 | 391 |

### Example 3

### Characterization of Physiological Phenotypes of Transgenic Plants Expressing a Heterologous Flavohemoglobin Protein

The physiological efficacy of transgenic corn plants (tested as hybrids) can be tested for nitrogen use efficiency (NUE) traits in a high-throughput nitrogen (N) screen. The collected data are compared to the measurements from wildtype controls using a statistical model to determine if the changes are due to the transgene. Raw data were analyzed by SAS software. Results shown herein are the comparison of transgenic plants relative to the wildtype controls.

### (1) Media Preparation for Planting a NUE Protocol

Planting materials used: Metro Mix 200 (vendor: Hummert) Cat. #10-0325, Scotts Micro Max Nutrients (vendor: Hummert) Cat. #07-6330, OS 4⅓″×3⅞″ pots (vendor: Hummert) Cat. #16-1415, OS trays (vendor: Hummert) Cat. #16-1515, Hoagland's macronutrients solution, Plastic 5″ stakes (vendor: Hummert) yellow Cat. #49-1569, white Cat. #49-1505, Labels with numbers indicating material contained in pots. Fill 500 pots to rim with Metro Mix 200 to a weight of ˜140 g/pot. Pots are filled uniformly by using a balancer. Add 0.4 g of Micro Max nutrients to each pot. Stir ingredients with spatula to a depth of 3 inches while preventing material loss.

### (2) Planting a NUE Screen in the Greenhouse

a. Seed Germination

Lightly water each pot twice using reverse osmosis purified water. The first watering should occur just before planting, and the second watering should occur after the seed has been planted in the pot. Ten Seeds of each entry (1 seed per pot) are planted to select eight healthy uniform seedlings. Additional wildtype controls are planted for use as border rows. Alternatively, 15 seeds of each entry (1 seed per pot) are planted to select 12 healthy uniform seedlings (this larger number of plantings is used for the second, or confirmation, planting). Place pots on each of the 12 shelves in the Conviron growth chamber for seven days. This is done to allow more uniform germination and early seedling growth. The following growth chamber settings are 25° C./day and 22° C./night, 14 hours light and ten hours dark, humidity ˜80%, and light intensity ˜350 μmol/m²/s (at pot level). Watering is done via capillary matting similar to greenhouse benches with duration of ten minutes three times a day.

b. Seedling Transfer

After seven days, the best eight or 12 seedlings for the first or confirmation pass runs, respectively, are chosen and transferred to greenhouse benches. The pots are spaced eight inches apart (center to center) and are positioned on the benches using the spacing patterns printed on the capillary matting. The Vattex matting creates a 384-position grid, randomizing all range, row combinations. Additional pots of controls are placed along the outside of the experimental block to reduce border effects.

Plants are allowed to grow for 28 days under the low N run or for 23 days under the high N run. The macronutrients are dispensed in the form of a macronutrient solution (see composition below) containing precise amounts of N added (2 mM NH₄NO₃ for limiting N screening and 20 mM NH₄NO₃ for high N screening runs). Each pot is manually dispensed 100 ml of nutrient solution three times a week on alternate days starting at eight and ten days after planting for high N and low N runs, respectively. On the day of nutrient application, two 20 min waterings at 05:00 and 13:00 are skipped. The vattex matting should be changed every third run to avoid N accumulation and buildup of root matter.

TABLE 11
This table shows the amount of nutrients in the
nutrient solution for either thelow or high nitrogen screen.

| | 2 mM NH₄NO₃ | 20 mM NH₄NO₃ (high |
|---|---|---|
| | (Low Nitrogen Growth | Nitrogen Growth |
| | Condition, Low N) | Condition, High N) |
| Nutrient Stock | mL/L | mL/L |
| 1M NH₄N0₃ | 2 | 20 |
| 1M KH₂PO₄ | 0.5 | 0.5 |
| 1M MgSO₄•7H₂O | 2 | 2 |
| 1M CaCl₂ | 2.5 | 2.5 |
| 1M K₂SO₄ | 1 | 1 |

Note:
Adjust pH to 5.6 with HCl or KOH

c. Harvest Measurements and Data Collection

After 28 days of plant growth for low N runs and 23 days of plant growth for high N runs, the following measurements are taken (phenocodes in parentheses): total shoot fresh mass (g) (SFM) measured by Sartorius electronic balance, V6 leaf chlorophyll measured by Minolta SPAD meter (relative units) (LC), V6 leaf area (cm²) (LA) measured by a Li-Cor leaf area meter, V6 leaf fresh mass (g) (LFM) measured by Sartorius electronic balance, and V6 leaf dry mass (g) (LDM) measured by Sartorius electronic balance. Raw data were analyzed by SAS software. Results shown are the comparison of transgenic plants relative to the wildtype controls.

To take a leaf reading, samples were excised from the V6 leaf. Since chlorophyll meter readings of corn leaves are affected by the part of the leaf and the position of the leaf on the plant that is sampled, SPAD meter readings were done on leaf six of the plants. Three measurements per leaf were taken, of which the first reading was taken from a point one-half the distance between the leaf tip and the collar and halfway from the leaf margin to the midrib while two were taken toward the leaf tip. The measurements were restricted in the area from ½ to ¾ of the total length of the leaf (from the base) with approximately equal spacing between them. The average of the three measurements was taken from the SPAD machine.

The characterization of physiological phenotypes according to the procedure disclosed above was carried out for corn transgenic lines comprising SEQ NO: 3 including ZM_M21516, ZM_M21505, ZM_M20388 and ZM_M21509.

TABLE 12
Increased chlorophyll level in transgenic corn plant comprising
the E. coli HMP gene grown under the limiting nitrogen condition
Chlorophyll (SPAD) results for corn plants grown under limiting nitrogen condition

| | Run 1 | | | | Run 2 | | | |
|---|---|---|---|---|---|---|---|---|
| Transgenic | | | | % | | | | % |
| Event | Transgenic | Control | Difference | Difference | Transgenic | Control | Difference | Difference |
| 20388 | 25.1 | 23.4 | 1.7 | 7 (a) | 25.1 | 23.8 | 1.3 | 6 (b) |
| 21505 | 25.1 | 23.4 | 1.70 | 7 (a) | 27.2 | 23.8 | 3.3 | 14 (a) |
| 21509 | ND | ND | ND | ND | ND | ND | ND | ND |
| 21516 | ND | ND | ND | ND | ND | ND | ND | ND |
| | Run 3 | | | | Run 4 | | | |
| Transgenic | | | | % | | | | % |
| Event | Transgenic | Control | Difference | Difference | Transgenic | Control | Difference | Difference |
| 20388 | 28.6 | 27.6 | 1.0 | 4 (n) | 22.7 | 21.3 | 1.4 | 7 (b) |
| 21505 | 31.3 | 27.6 | 3.7 | 13 (a) | 22.8 | 21.3 | 1.5 | 7 (b) |
| 21509 | 29.4 | 27.6 | 1.8 | 6 (b) | 22.8 | 21.3 | 1.5 | 7 (b) |
| 21516 | 28.7 | 27.6 | 1.1 | 4 (n) | 22.7 | 21.3 | 1.4 | 7 (b) |

(a): highly significant, p < 0.01 in the current dataset
(b): significant, 0.01 < p < 0.05 in the current dataset
(c): significant, 0.05 < p < 0.1 in the current dataset
(n): non-significant, p > 0.1 in the current dataset
ND: not determined in the current dataset

TABLE 13
Increased shoot fresh mass in transgenic comprising
the E. coli HMP gene under the sufficient nitrogen condition
Shoot Fresh Mass Results for transgenic plants grown under the sufficient nitrogen condition

| | Run 1 | | | | Run 2 | | | |
|---|---|---|---|---|---|---|---|---|
| Transgenic | Transgenic | Control | Difference | % | Transgenic | Control | Difference | % |
| Event | (g) | (g) | (g) | Difference | (g) | (g) | (g) | Difference |
| 20388 | 68.7 | 54.8 | 13.9 | 25 (a) | 87.8 | 86.3 | 1.5 | 2 (n) |
| 21505 | 65.6 | 54.8 | 10.8 | 20 (a) | 94.3 | 86.3 | 7.9 | 9 (n) |
| 21509 | 56.4 | 54.8 | 1.6 | 3 (n) | 100.8 | 86.3 | 14.5 | 17 (b) |
| 21516 | 40.9 | 54.8 | 13.9 | −25 (a) | 120.3 | 86.3 | 33.9 | 39 (a) |
| | Run 3 | | | | Run 4 | | | |
| Transgenic | Transgenic | Control | Difference | % | Transgenic | Control | Difference | % |
| Event | (g) | (g) | (g) | Difference | (g) | (g) | (g) | Difference |
| 20388 | 63.1 | 55.1 | 8.0 | 15 (a) | 58.8 | 52.2 | 6.6 | 13 (c) |
| 21505 | 56.9 | 55.1 | 1.8 | 3 (n) | 68.1 | 52.2 | 15.9 | 31 (a) |
| 21509 | ND | ND | ND | ND | 61.6 | 52.2 | 9.4 | 18 (b) |
| 21516 | ND | ND | ND | ND | 61.8 | 52.2 | 9.7 | 19 (b) |

(a): highly significant, p < 0.01 in the current dataset
(b): significant, 0.01 < p < 0.05 in the current dataset
(c): significant, 0.05 < p < 0.1 in the current dataset
(n): non-significant, p > 0.01 in the current dataset
ND: not determined in the current dataset

### Example 4

### Characterization of Plant Yield

Of particular interest is the identification of transgenic plants having improved yield as the result of enhanced seed sink potential and/or strength. The sink approach includes strategies to enhance sink potential (the number and size of endosperm cells or of kernels) and to enhance sink strength (the rate of starch biosynthesis). Sink potential can be established very early during kernel development, as endosperm cell number and cell size are determined within the first few days after pollination. Carbon flow to the ear during development may be limited by the size of the grain sink. Improvements in sink strength have been suggested to enhance yield by promoting the redistribution of photoassimilate from stem to kernel tissue.

Much of the increase in corn yield of the past several decades has resulted from an increase in planting density. During that period, corn yield has been increasing at a rate of 2.1 bushels/acre/year, but the planting density has increased at a rate of 250 plants/acre/year. A characteristic of modern hybrid corn is the ability of these varieties to be planted at high density. Many studies have shown that a higher than current planting density should result in more biomass production, but current germplasm does not perform well at these higher densities. One approach to increasing yield is to increase harvest index (HI), the proportion of biomass that is allocated to the kernel compared to total biomass, in high density plantings.

The ability of a plant to convert CO₂ and light into carbon which can be exported to developing seeds is known as source potential. Several lines of genetic, physiological and biochemical evidence suggest that source potential is a direct contributor to yield. Approaches to increase source potential, and thus yield by enhancing net carbon assimilation include increasing intrinsic photosynthetic efficiency, altering the partitioning and export of assimilates, and modifying plant architecture. Genes that can change these properties in a beneficial manner have been identified and introduced into plants.

The design of yield testing by the present invention is a high throughput hybrid yield screening process. It is based on two year complementary multi-location testing. Both Year 1 and Year 2 trials are multi location, single rep per location experiments arranged using spatially based experimental design. All trials at different locations are grown under optimal production management practices, and maximum pest control.

### (1) Year 1 Trial

Year 1 trial is the first level screen for yield where many transgenic events are expected to be tested using the approach mentioned above with moderate power (85%) to detect 7.5% yield difference. At each field location of up to 16 different geographic locations, events representing recombinant DNA constructs selected from the present invention, multiple positive and negative control plants, and pollinator plots are planted. The plot size is two row plots, 20 ft long×5 ft wide with 30 in distance between rows and three ft alley between ranges. Events grouped within constructs are randomly placed in the field. All other entries are also randomly placed in the field. A pollinator plot (LH244XLH59) is planted for every two plots of male sterile transgenic events. The planting density is approximately 28000-33000 plants/acre. The trial is open pollinated.

### (2) Year 2 Trial

Year 2 trial is confirmatory yield trial with events advanced based on Year 1 hybrid yield performance. Year 2 trials are designed to provide >80% power to detect 5-10% of yield difference. At each of up to 16 different geographic locations (or at least 20 growing environments), plots comprising events representing recombinant DNA constructs selected from the present inventions, multiple positive and negative control plants, and pollinator plots are planted. The plot size is two row plots, 20 ft long×5 ft wide with 30 in distance between rows and 3 ft alley between ranges. Events representing the same construct are grouped within construct block and that section randomly placed in the field. All other entries are also randomly placed in the field. A pollinator plot (LH244XLH59) is planted for every two plots of male sterile transgenic events. The planting density is approximately 28000 to 33000 plants/acre. The trial is open pollinated.

### (3) Statistical Method

This method comprises three major components: modeling spatial autocorrelation of the test field separately for each location, adjusting phenotypes of transgene-entries for spatial dependence for each location, and conducting an across location analysis and making gene advancement decisions. In addition, the method also has the capability to estimate the effects of different seed sources and adjust accordingly. This is done separately for each location when phenotypes of transgene-entries are adjusted for spatial dependence.

a. Modeling Spatial Autocorrelation

### Estimating the Covariance Parameters

Estimating the covariance parameters of the semivariogram is the first step. A spherical covariance model is assumed to model the spatial autocorrelation. Because of the size and nature of the trial, it is highly likely that the spatial autocorrelation may change. Therefore, anisotropy is also assumed along with spherical covariance structure. The following set of equations describes the statistical form of the anisotropic spherical covariance model.

$$C ( h ; θ ) = vI ( h = 0 ) + {σ}^{2} ( 1 - \frac{3}{2} h + \frac{1}{2} {h}^{3} ) I ( h < 1 ) ,$$

where I() is the indicator function,

h=\sqrt{\dot{x}²+\dot{y}²},

and

\dot{x}=[cos(ρπ/180)(x₁−x₂)−sin(ρπ/180)(y₁−y₂)]/ωₓ

\dot{y}=[sin(ρπ/180)(x₁−x₂)+cos(ρπ/180)(y₁−y₂)]/ω_{y}

where s₁=(x₁, y₁) are the spatial coordinates of one location and s₂=(x₂, y₂) are the spatial coordinates of the second location. There are 5 covariance parameters, θ=(v, σ², ρ, ωₙ, ωⱼ), where □ is the nugget effect, □² is the partial sill, □ is a rotation in degrees clockwise from north, □ₙ is a scaling parameter for the minor axis and □ⱼ is a scaling parameter for the major axis of an anisotropical ellipse of equal covariance.

The five covariance parameters that define the spatial trend will then be estimated by using data from heavily replicated pollinator plots via restricted maximum likelihood approach. In a multi-location field trial, the spatial trend is modeled separately for each location.

b. Building Variance-Covariance Matrix

After obtaining the variance parameters of the model, variance-covariance structure will be generated for the data set to be analyzed. This variance-covariance structure will contain the spatial information required to adjust transgene (unreplicated) yields for spatial dependence.

### Adjusting Transgene Data for Spatial Dependence

Adjusting the transgene data for spatial dependence is the next step. In this case, a nested model that best represents the treatment and experimental design of the study will be used along with the variance-covariance structure to adjust the yields of transgene-entries for spatial dependence. During this process the nursery or the seed batch effects can also be modeled and estimated to adjust the yields for any yield parity caused by seed batch differences.

### Combined Location Analysis

Spatially adjusted data from different locations are first generated. Then all the adjusted data will be combined and analyzed assuming locations as replications using the third phase of this method. In this analysis, intra and inter-location variances will be combined to estimate the standard error of the transgene and any associated treatment control data.

The yield analysis according to the procedure disclosed above was carried out for corn transgenic lines comprising SEQ NO: 3 including ZM_M21516, ZM_M21505, ZM_M20388 and ZM_M21509, and corn transgenic lines comprising SEQ NO: 4 including ZM_M14965, ZM_M16110, ZM_M16104 and ZM_M14973.

TABLE 14
Year 1yield results for transgenic corn plants comprising the E. coli HMP gene
2004 Yield for transgenic corn plants comprising the E. coli HMP gene

| | Transgenic | Control | Transgenic − | | | |
|---|---|---|---|---|---|---|
| Transgenic | Yield, | Yield, | Control, | Percent | | |
| Event | Bu/Ac | Bu/Ac | Bu/Ac | Difference | P-Value | Significance |
| ZM_M21516 | 237.6 | 226.2 | 11.4 | 5.10% | 0.0066 | Significant |
| ZM_M21505 | 216.8 | 226.2 | −9.4 | −4.10% | 0.0257 | Significant |
| ZM_M20388 | 223 | 226.2 | −3.1 | −1.40% | 0.4561 | Not significant |
| ZM_M21509 | 221.4 | 226.2 | −4.8 | −2.10% | 0.2515 | Not significant |

TABLE 15
Year 2 yield results for transgenic corn plants comprising the E. coli HMP gene
2005 Yield for transgenic corn plants comprising the E. coli HMP gene

| | Transgenic | Control | Transgenic − | | | |
|---|---|---|---|---|---|---|
| Transgenic | Yield, | Yield, | Control, | Percent | | |
| Event | Bu/Ac | Bu/Ac | Bu/Ac | Difference | P-Value | Significance |
| ZM_M21516 | 176.9 | 179.9 | −3.0 | −1.68% | 0.2909 | Not significant |
| ZM_M21505 | Not tested | / | / | / | / | / |
| ZM_M20388 | 179.1 | 179.9 | −0.8 | −0.45% | 0.7781 | Not significant |
| ZM_M21509 | 168.4 | 179.9 | −11.5 | −6.38% | 0 | Significant |

In 2004, the mean control yield was 226.2 bushels/acre versus 179.9 bushels/acre in 2005, the latter being a drought year. Reduced water uptake during drought conditions also restricts nutrient uptake from the soil solution hence confounding the yield response. Thus, differences in yield potential and growing conditions from 2004 to 2005 do not allow a valid comparison of the yield response, but provide a clue to the gene effect with environment interaction.

TABLE 16
Yield 1 yield results for transgenic corn plants comprising the Yeast YHB1 gene
2003 Yield for transgenic corn plants comprising the Yeast YHB1 gene

| | Transgenic | Control | Transgenic − | | | |
|---|---|---|---|---|---|---|
| Transgenic | Yield, | Yield, | Control, | Percent | | |
| Event | Bu/Ac | Bu/Ac | Bu/Ac | Difference | P-Value | Significance |
| ZM_M14965 | 154.4 | 155.4 | −1 | −1% | 0.8731 | Not significant |
| ZM_M16110 | 159.7 | 155.4 | 4.3 | 3% | 0.4995 | Not significant |
| ZM_M16104 | 158.5 | 155.4 | 3.2 | 2% | 0.6551 | Not significant |
| ZM_M14973 | 148.1 | 155.4 | −7.3 | −5% | 0.2757 | Not significant |

TABLE 17
Year 2 yield results for transgenic corn plants comprising the Yeast YHB1 gene
2004 Yield for transgenic corn plants comprising the Yeast YHB1 gene

| | Transgenic | Control | Transgenic − | | | |
|---|---|---|---|---|---|---|
| Transgenic | Yield, | Yield, | Control, | Percent | | |
| Event | Bu/Ac | Bu/Ac | Bu/Ac | Difference | P-Value | Significance |
| ZM_M14965 | 225.8 | 218.7 | 7.1 | 3.20% | 0.0068 | Significant |
| ZM_M16110 | 223.3 | 218.7 | 4.6 | 2.10% | 0.0775 | Significant |
| ZM_M16104 | 220.6 | 218.7 | 1.9 | 0.90% | 0.4598 | Not significant |
| ZM_M14973 | 217.4 | 218.7 | −1.3 | −0.60% | 0.6281 | Not significant |

Overall, in 2004, the environment in the locations of field yield testing was more favorable for yield production than that in 2003, which may account for the difference in yield performance of transgenic plants comprising SEQ ID NO: 4.

### Example 5

### E. coli HMP Reduces the NO Level in Plants

A confocal microscopy analysis was carried out to detect the NO levels in transgenic corn plants comprising the E. coli HMP gene using a NO-specific dye named DAF-2DA (Calbiochem). DAF-2DA is the most sensitive reagent available for detecting NO: its detection limit is 5 nM, two orders of magnitude lower than next best method, paramagnetic resonance spectroscopy. Four maize events, namely ZM_M21505, ZM_M21516, ZM_M20388, ZM_M 21509 and nontransgenic controls were planted in the greenhouse under standard maize growing conditions. 12 plants/event were grown in the presence of either the limiting nitrogen growth condition (2 mM ammonium nitrate) or the sufficient nitrogen growth condition (20 mM ammomium nitrate). Additionally, border plants were included in the experiment to ensure homogeneity in growth conditions. The plants were randomized using Virgo's Make-a-map program. When plants reached V6 stage, 2×2 inch leaf samples were harvested from the terminal segment of the leaf blade and immediately incubated in Tris 10 mM pH=7 during transportation to the microscopy lab. At least ten very thin (one mm wide) sections per sample were then generated from each harvested leaf and incubated in dark in 10 micromolar DAF-2DA in distilled water for 1 hour under gentle shaking. NO levels were visualized using a confocal laser scanning microscope (Zeiss LSM510). Images were processed using the Zeiss LSM Image Browser. On average, three plants per event were analyzed along with controls grown under the same conditions. In all four events, transgenic plants grown under limiting or sufficient nitrogen showed lower levels of NO compared to controls grown under the same conditions.

This experiment also allowed for the exploration of the spatial expression of NO in corn plants. Under either the sufficient or the limiting nitrogen growth condition, the DAF-2DA staining signals were localized in bundle sheath cells and mesophyll cells of control plants, suggesting that these cells are involved in NO metabolism and also that they contain the required esterases for activation of DAF2-DA. It was also observed a reduction of DAF-2DA signal in bundle sheath cells and mesophyll cells in transgenic corn plants comprising the E. coli HMP gene, which is consistent with the expected molecular activity of flavohemoglobin and the expected pattern of transgene expression driven by the rice actin promoter in these cell types. In addition, the histogram function provided by the Carl Zeiss LSM Image Examiner was used to quantify the NO-specific signals. We demonstrated a decrease in DAF2-DA staining intensity in transgenics vs. controls in five plants belonging to event ZM_M21516.

TABLE 18
Percent decrease in DAF2-DA staining intensity
in five transgenic plants of event ZM_M21516

| | % decrease |
|---|---|
| | in transgenic corn |
| | plants as compared to |
| ZM_M21516 lines tested | controls |
| Transgenic plant 1 vs. control 1 | 31.01773 |
| Transgenic plant 2 vs. control 2 | 52.75593 |
| Transgenic plant 3 vs. control 3 | 40.91894 |
| Transgenic plant 4 vs. control 4 | 52.41726 |
| Transgenic plant 5 vs. control 5 | 78.37197 |
| Average of Decrease | 51.09636 |
| St. Dev. | 17.71433 |

### Example 6

### Analysis of Free Amino Acid Content in Transgenic Corn Plants Comprising the E. coli HMP Gene

Transgenic events and non-transgenic controls were grown under sufficient nitrogen fertilized with 225 lbs. N/Ac. When plants reached stage V12, the ear leaf was removed from 12 plants each of wild-type or either transgenic events, then analyzed for free amino acids.

Samples were prepared accurately weighing approximately 50 mg of homogenous dry powder and extracting it with 1.5 ml of a 10% w/v TCA solution. The sample was clarified by centrifugation and 0.5 ul of supernatant was analyzed for free amino acids. The HPLC system consisted of an Agilent 1100 HPLC with a cooled autosampler, a fluorescence detector, and a HP Chemstation data system. Separation of the amino acids was performed using precolumn o-phthalaldehyde (OPA) derivatization followed by separation using a Zorbax Eclipse-AAA 4.6×75 mm, 3.5 um column. Detection was by fluorescence and chromatograms were collected using the HP Chemstation. All standards and reagents were purchased from Agilent Technologies.
Ref: Rapid, Accurate, Sensitive and Reproducible Analysis of Amino Acids; John W. Henderson, Robert D. Ricker, Brian A. Bidlingmeyer, Cliff Woodward, Agilent publication 5980-1193EN

TABLE 19
Free amino acid levels in corn plant leaves

| Amino | Wild-type | Event ZM_M21505 | | Event ZM_M 20388 | |
|---|---|---|---|---|---|
| Acid | PPM | PPM | % Change | PPM | % Change |
| Ala | 2329 | 2812 | 20.7 (a) | 1878 | −19.4 (a) |
| Glu | 1741 | 2301 | 32.2 (a) | 1396 | −19.8 (a) |
| Ser | 369 | 510 | 38.2 (a) | 296 | −19.8 (n) |
| Gln | 218 | 329 | 50.9 (a) | 144 | −33.9 (a) |
| Thr | 157 | 212 | 35.0 (a) | 116 | −26.1 (n) |
| Arg | 119 | 77 | −35.3 (n) | 65 | −45.4 (a) |
| Gly | 119 | 185 | 55.5 (a) | 37 | −68.9 (a) |
| Asn | 114 | 246 | 115.8 (a) | 52 | −54.4 (a) |
| Asp | 108 | 56 | −48.1 (n) | 84 | −22.2 (n) |
| Val | 9 | 0 | 0 | 0 | 0 |
| Tyr | 3.3 | 0 | 0 | 0 | 0 |
| His | 0 | 0 | 0 | 0 | 0 |
| Ile | 0 | 0 | 0 | 0 | 0 |
| Leu | 0 | 0 | 0 | 0 | 0 |
| Lys | 0 | 0 | 0 | 0 | 0 |
| Met | 0 | 0 | 0 | 0 | 0 |
| Phe | 0 | 0 | 0 | 0 | 0 |
| Trp | 0 | 0 | 0 | 0 | 0 |
| Total | 5286.3 | 6728 | 27.3 (a) | 4068 | −23.0 (a) |

(a): Significant, p < 0.05 in the current dataset
(n): non-significant in the current dataset
0: not detected in the current dataset

### Example 7

### Identification of Homologs

A BLAST searchable “All Protein Database” was constructed of known protein sequences using a proprietary sequence database and the National Center for Biotechnology Information (NCBI) non-redundant amino acid database (nr.aa). For E. Coli, from which the polynucleotide sequence as set forth in SEQ ID NO:1 was obtained, an “Organism Protein Database” was constructed of known protein sequences of the organism. The Organism Protein Database is a subset of the All Protein Database based on the NCBI taxonomy ID for the organism.

The All Protein Database was queried using the amino acid sequence as set forth in SEQ ID NO: 5 by “blastp” with an E-value cutoff of 1e-8. Up to 1000 top hits were kept, and separated by organism names. For each organism other than E. coli, a list was kept for hits from the query organism itself with a more significant E-value than the best hit of the organism. The list contains likely duplicated genes, and is referred to as the Core List. Another list was kept for all the hits from each organism, sorted by E-value, and referred to as the Hit List.

The Organism Protein Database was queried using the amino acid sequence as set forth in SEQ ID NO: 5 using “blastp” with E-value cutoff of 1e-4. Up to 1000 top hits were kept. A BLAST searchable database was constructed based on these hits, and is referred to as “SubDB”. SubDB was queried with each sequence in the Hit List using “blastp” with E-value cutoff of 1e-8. The hit with the best E-value was compared with the Core List from the corresponding organism. The hit is deemed a likely ortholog if it belongs to the Core List, otherwise it is deemed not a likely ortholog and there is no further search of sequences in the Hit List for the same organism. Likely orthologs from a large number of distinct organisms were identified and are reported by amino acid sequences of SEQ ID NO: 130 to SEQ ID NO: 256.

All patents, patent applications and publications cited herein are incorporated by reference in their entirety to the same extent as if each individual patent, patent application or publication was specifically and individually indicated to be incorporated by reference.

What is claimed is:

1. A non-naturally occurring DNA comprising a polynucleotide sequence selected from the group consisting of SEQ ID NO: 1, 2 and 260, and complement thereof.

2. A recombinant DNA construct for plant transformation comprising a polynucleotide selected from the group consisting of SEQ ID NO: 1, 2 and 260.

3. The recombinant DNA construct according to claim 2 that further comprises a promoter for plant expression.

4. The recombinant DNA construct according to claim 3, wherein said promoter is selected from a group consisting of a constitutive promoter, a green tissue preferred promoter, a phloem preferred promoter and a root tissue preferred promoter.

5. Transgenic seed comprising a heterologous flavohemoglobin gene in its genome, wherein transgenic plants grown from said transgenic seed exhibit an improved agronomic trait, as compared to a control plant.

6. Transgenic seed according to claim 5, wherein said flavohemoglobin gene encodes a protein having an amino acid sequence selected from the group consisting of SEQ ID NO: 5 and 6, and homologs thereof.

7. Transgenic seed according to claim 6, wherein said homolog has an amino acid sequence selected from the group consisting of SEQ ID NO: 130 through SEQ ID NO: 256.

8. Transgenic seed according to claim 5, wherein said improved agronomic trait is a:
(a) faster rate of growth,
(b) increased fresh or dry biomass,
(c) increased seed or fruit yield,
(d) increased seed or fruit nitrogen content,
(e) increased free amino acid content in whole plant,
(f) increased free amino acid content in seed or fruit,
(g) increased protein content in seed or fruit,
(h) increased chlorophyll level, and/or
(i) increased protein content in vegetative tissue.

9. Transgenic seed according to claim 5, wherein said transgenic plants having an improved agronomic trait are grown under a sufficient nitrogen growth condition or a limiting nitrogen growth condition.

10. A method of producing a transgenic plant having an improved agronomic trait, wherein said method comprises
(a) transforming plant cells with a recombinant DNA construct for expressing a flavohemoglobin protein;
(b) regenerating plants from said cells; and
(c) screening said plants to identify an improved agronomic trait.

11. The method according to claim 10, wherein said improved agronomic trait is a:
(a) faster rate of growth,
(b) increased fresh or dry biomass,
(c) increased seed or fruit yield,
(d) increased seed or fruit nitrogen content,
(e) increased free amino acid content in whole plant,
(f) increased free amino acid content in seed or fruit,
(g) increased protein content in seed or fruit,
(h) increased chlorophyll level, and/or
(i) increased protein content in vegetative tissue.

12. The method according to claim 10, wherein said transgenic plants are grown under a sufficient nitrogen growth condition or a limiting nitrogen growth condition.

13. The method according to claim 10, wherein said recombinant DNA construct comprises a polynucleotide encoding a protein having an amino acid sequence selected from the group consisting of SEQ ID NO: 5 and 6, and homologs thereof.

14. The method according to claim 13, wherein said homolog has an amino acid sequence selected from the group consisting of SEQ ID NO: 130 through SEQ ID NO: 256.

15. The method according to claim 14, wherein said recombinant DNA construct further comprises a promoter for plant expression.

16. The method according to claim 15, wherein said promoter selected from the group consisting of a constitutive promoter, a root preferred promoter, a phloem preferred promoter and a green tissue preferred promoter.

17. The method according to claim 16, wherein said constitutive promoter is rice actin promoter.

18. The method according to claim 16, wherein said root preferred promoter is rice RCC3 promoter.

19. The method according to claim 16, wherein said green tissue preferred promoter is FDA or PPDK promoter.

20. The method according to claim 16, wherein said phloem preferred promoter is RTBV promoter.
